(19) United States
(12) Patent Application Publication
(10) Pub. No.: US 2021/0206788 A1
(43) Pub. Date: Jul. 8, 2021

(54) IONIC METAL ALKYLIDENE COMPOUNDS AND USE THEREOF IN OLEFINIC METATHESIS REACTIONS

(71) Applicant: Verbio Vereinigte Bioenergie AG, Zörbig (DE)

(72) Inventors: Levente Ondi, Budapest (HU); Roman Schowner, Stuttgart (DE); Michael R. Buchmeiser, Stuttgart (DE); Iris Elser, Stuttgart (DE); Florian Toth, Budapest (HU); Emmanuel Robe, Budapest (HU); Benedek Vakulya, Budapest (HU)

(21) Appl. No.: 17/057,864

(22) PCT Filed: May 31, 2019
(86) PCT No.: PCT/EP2019/064192

§ 371 Date: Nov. 23, 2020

(30) Foreign Application Priority Data
Jun. 1, 2018 (EP) 18175620.6

(51) Int. Cl. C07F 11/00; C07B 37/10; B01J 31/22; C07F 5/02

(52) U.S. Cl. CPC C07F 11/00; C07B 37/10; B01J 31/2295; B01J 2231/543; B01J 2531/64; B01J 2531/66; C07F 5/027

(57) ABSTRACT

A compound of formula (I) wherein: M is selected from Mo or W; X is selected from O or NR⁵; R¹ and R² are independently selected from H, C₁₋₆ alkyl, and aryl; C₁₋₆ alkyl and aryl optionally being substituted with one or more of C₁₋₆ alkyl, C₁₋₆ alkoxy, and O—C₆H₅; R³ is selected from a nitrogen-containing aromatic heterocycle being bound to M via said nitrogen; and from halogen; R⁴ is an aryl oxy group being bound to M via said oxygen of said aryl oxy group; wherein said aryl group Ar of said aryl oxy group is bound to a group Cat such to form a cationic ligand Cat⁺-Z—ArO—, wherein Z is either a covalent bond or a linker; R⁵ is alkyl or aryl, optionally substituted.

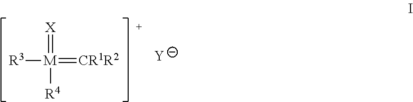

### FIELD OF THE INVENTION

The present invention relates to ionic metal alkylidene compounds and use thereof as catalysts in metathesis reactions. The invention further relates to a method of making the compounds and to a composition comprising same.

### BACKGROUND OF THE INVENTION

Olefinic metathesis using metal alkylidene catalysts such as Schrock catalysts is considered one of the most useful C—C coupling reactions. Apart from functional group tolerance, high activity, and high productivity, the synthesized products should be available with low metal contamination stemming from the catalyst.

Elser I. et al.: “Molybdenum and Tungsten Imido Alkylidene N-Heterocyclic Carbene Catalysts Bearing Cationic ligands for Use in Biphasic Olefin Metathesis”, Chem. Eur. 2017, 23, 6398-6405, suggest using molybdenum and tungsten imido alkylidene complexes bearing a cationic ligand, and to conduct a biphasic olefin metathesis using pyrrole and a hydrocarbon as solvents in order to avoid contamination. Since it is known from other cationic molybdenum imido, tungsten imido, and tungsten oxo alkylidene N-heterocyclic carbene complexes that the high reactivity in standard olefin metathesis reactions is considerably weakened when using NHC-free counterparts, said ionic catalysts defined in the reference are consequently ligated with a N-heterocyclic carbene (NHC) ligand in order to promote and ensure reactivity.

### OBJECTS OF THE INVENTION

Due to the growing importance of metathesis catalysts there is an ongoing need in the industry for such catalysts which achieve high turnover numbers, which are stable under the reaction conditions, which tolerate functional groups in the olefins to be subjected to metathesis, and which allow the synthesis of products having a low or even no metal contamination.

### SUMMARY OF THE INVENTION

This object has been achieved with compounds of formula I

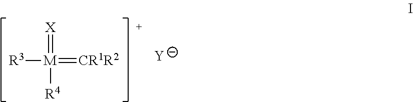

as defined in independent claim 1.

The compounds of formula I may be regarded as the NHC-free counterparts of the catalysts as referred to in the reference mentioned in the Background section. It could not be expected in view of the teaching of this prior art regarding the crucial importance of a NHC ligand that despite the absence of a NHC ligand in the compounds of formula I the object could be achieved.

Moreover, the new catalysts may provide for Z-selectivity, i.e. they may favour the formation of Z-olefins over the formation of E-olefins. This is also not derivable from the reference. This property is of benefit since the use of Z-olefins is frequently required in chemical syntheses.

Preferred embodiments are defined in the claims depending on claim 1.

This object has also been achieved with a compound of formula IV

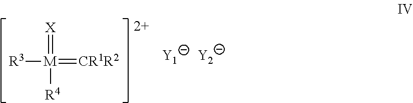

as defined in this disclosure.

The object has been further achieved with compounds of formula VII

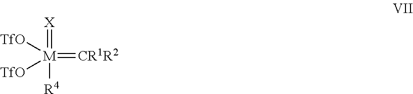

as defined in this disclosure.

### DETAILED DESCRIPTION OF THE INVENTION

In a first aspect, the invention relates to a metal alkylidene compound of formula I

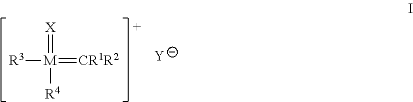

wherein:
M is selected from Mo or W;
X is selected from O or NR⁵;
R¹ and R² are independently selected from H, C₁₋₆ alkyl, and aryl; C₁₋₆ alkyl and aryl optionally being substituted with one or more of C₁₋₆alkyl, C₁₋₆ alkoxy, and O—C₆H₅;
R³ is selected from a nitrogen-containing aromatic heterocycle being bound to M via said nitrogen; halogen; and triflate;
R⁴ is an aryl oxy group being bound to M via said oxygen of said aryl oxy group; wherein said aryl group Ar of said aryl oxy group is bound to a group Cat such to form a cationic ligand Cat⁺-Z—ArO—, wherein Z is either a covalent bond or a linker;
R⁵ is alkyl or aryl, optionally substituted; and
Y^{⊖} is a non-nucleophilic anion.

According to the invention, M is selected from Mo and W.

Further according to the invention, X is selected from O or NR⁵. Thus, the alkylidene compounds according to the invention encompass metal oxo alkylidene compounds and metal imido alkylidene compounds.

Further according to the invention, R¹ and R² of the alkylidene moiety are independently selected from H, C₁₋₆ alkyl, and aryl, wherein alkyl and aryl may optionally be substituted.

The term “alkyl” as used herein encompasses linear, branched and cyclic alkyl.

The term “aryl” as used herein encompasses phenyl and naphthyl.

In one embodiment, the optional substituents are selected from C₁₋₆ alkyl, C₁₋₆ alkoxy, and O—C₆H₅.

Preferred ligands R¹ and R² are independently selected from H, C(CH₃)₃, and C(CH₃)₂C₆H₅.

A further preferred ligand is C₆H₅.

In one embodiment, when one of R¹ and R² is phenyl, phenyl is optionally substituted in o-position with C₁₋₆ alkoxy or O—C₆H₅

In a further preferred embodiment, one of R¹ and R² is H, and the other is C(CH₃)₃, C(CH₃)₂C₆H₅, or phenyl optionally substituted in o-position with C₁₋₆ alkoxy or O—C₆H₅.

Further according to the invention, in one embodiment, R³ is selected from a nitrogen-containing aromatic heterocycle being bound to M via said nitrogen.

In a preferred embodiment, R³ is selected from pyrrol-1-yl, pyrazol-1-yl, imidazol-1-yl, 1H-1,2,3-triazol-1-yl, 2H-1,2,3-triazol-2-yl, 1H-1,2,4-triazol-1-yl, 4H-1,2,4-triazo-4-yl, indol-1-yl, indazol-1-yl, and azaindol-1-yl, optionally substituted with one or more substituents selected independently from C₁₋₆ alkyl, C₁₋₆ alkoxy, phenyl, halogen, or cyano.

In a preferred embodiment, R³ is selected from pyrrol-1-yl, 2,5-dimethylpyrrol-1-yl, and 2,5-diphenylpyrrol-1-yl.

In another preferred embodiment, R³ is selected from indol-1-yl, optionally substituted with one or more substituents selected independently from C₁₋₆ alkyl, C₁₋₆ alkoxy, phenyl, halogen, or cyano.

Further according to the invention, in one embodiment, R³ is selected from halogen, preferably chlorine or bromine, more preferred chlorine.

Further according to the invention, in one embodiment, R³ is selected from triflate (CF₃SO₂O—).

Accordingly, the compounds according to the invention encompass both pyrrolido complexes, halogeno complexes, and triflate complexes.

Effective metathesis Schrock alkylidene catalysts typically contain an aryl oxy moiety bound to M via the oxygen atom of the aryl oxy moiety.

In the compounds according to the invention of formula I, said respective ligand R⁴ is an aryl oxy group being bound to M via said oxygen of said aryl oxy group; wherein said aryl group Ar of said aryl oxy group is bound to a group Cat such to form a cationic ligand Cat⁺-Z—ArO—, wherein Z is either a covalent bond or a linker.

The term “group Cat” as used herein encompasses any group capable of bearing a positive charge or being transferred to a positively charged condition when linked to the Z-aryl moiety of the aryl oxy ligand.

In one embodiment, said group Cat is directly attached to the aryl group.

The term “direct” as used herein means that the atom which carries the positive charge of the group Cat is bound to the aryl moiety of the aryl oxy ligand via a covalent bond, i.e. Z is a covalent bond.

In another embodiment, said aryl oxy moiety bears a substituent which in turn bears the group Cat.

Accordingly, the group Cat is indirectly attached to the aryl group.

The term “indirect” as used herein means that the atom which carries the positive charge of the cationic group is bound or connected to the aryl moiety of the aryl oxy ligand via a linker or spacer, i.e. Z is a linker.

The term “linker” is synonymously used with the term “spacer”.

Suitable linkers or spacers are known in the art. Exemplary linkers are alkylene chains, alkenylene chains, oxo alkylene chains, or aryl rings. Suitable aryl rings are e.g. phenyl, naphthyl, or biphenyl.

In one embodiment, said group Cat forms together with Z—ArO— a group Cat⁺-Z—ArO— selected/derived from an ammonium, pyridinium, phosphonium, phosphorinium, arsonium, sulfonium, and oxo sulfonium group.

In a preferred embodiment, said R⁴ is a pyridinium N-phenoxy group or a phosphonium P-phenoxy group.

The term “pyridinium N-phenoxy group” as used herein means that the pyridinium moiety bearing the positive charge is bound to the aryl group via said nitrogen and via a covalent bond.

The term “phosphonium P-phenoxy group” as used herein means that the phosphonium moiety bearing the positive charge is bound to the aryl group via said phosphorus and via a covalent bond.

A preferred phosphonium P-phenoxy group is triphenylphosphonium P-phenoxy.

In a preferred embodiment, said Ar in said Cat⁺-Z—ArO— is phenyl substituted in 2,6-position (with respect to O) with aryl or heteroaryl, respectively, preferably phenyl, optionally substituted.

The optional substituents of said aryl or phenyl may be independently selected from C₁₋₁₀ alkyl, optionally substituted with halogen such as fluorine, C₁₋₁₀ alkoxy, halogen, nitro, cyano, phenyl, phenoxy, N(C₁₋₆alkyl)₂, C(O)N(C₁₋₆alkyl)₂, C(O)NH(C₁₋₆ alkyl), C(O)O—C₁₋₆ alkyl, and two or more thereof.

In a further preferred embodiment, said Ar in said Cat⁺-Z—ArO— is phenyl substituted in 2,6-position with iso-propyl or t-butyl, respectively.

In a further preferred embodiment, said Ar in said Cat⁺-Z—ArO— is phenyl substituted in 4-position (with respect to 0) with Cat⁺-Z—.

In a further preferred embodiment, said Ar in said Cat⁺-Z—ArO— is phenyl substituted in 2,6-position with aryl or heteroaryl, or iso-propyl or t-butyl, respectively, and is substituted in 4-position with Cat⁺-Z—.

In a preferred embodiment R⁴=Cat⁺-Z—ArO— is selected from the group consisting of:

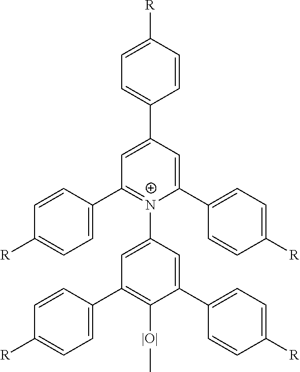

wherein R is H, C(CH₃)₃, C₆H₅, CF₃ or C₆F₁₃;

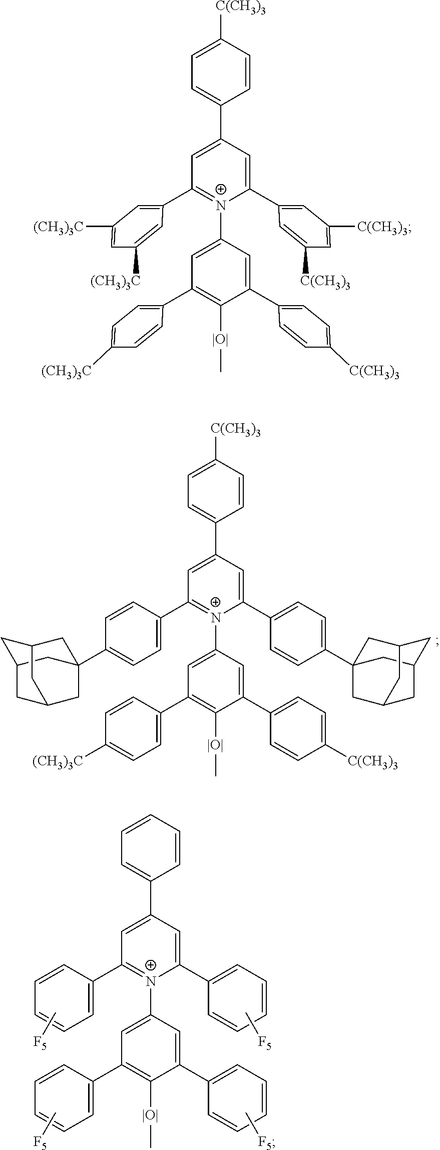

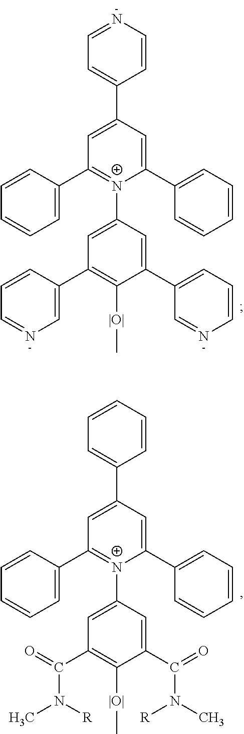

wherein R is H or CH₃;

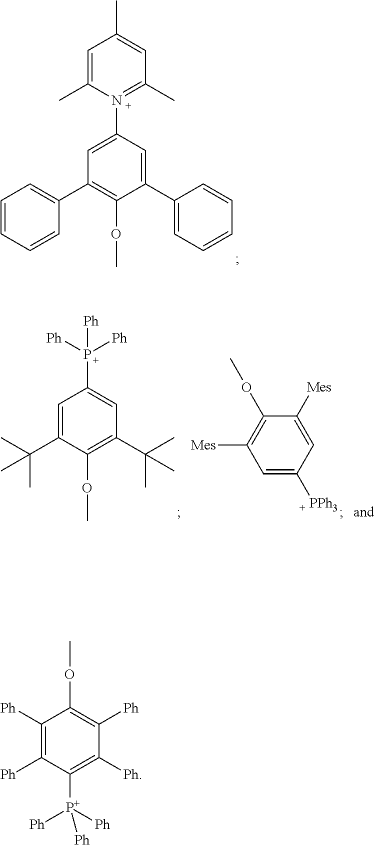

In another preferred embodiment, the term “pyridinium N-phenoxy group” as used herein means that the pyridinium moiety bearing the positive charge is bound to the aryl group via said nitrogen and via a linker.

In another preferred embodiment, the term “phosphonium P-phenoxy group” as used herein means that the phosphonium moiety bearing the positive charge is bound to the aryl group via said phosphorus and via a linker.

A group Cat⁺-Z—ArO— in which Z is a linker is e.g. a pyridinium styryl system of formula

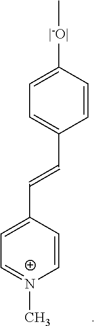

Another preferred group Cat⁺-Z—ArO— in which Z is an aryl linker is e.g. a substituted or unsubstituted phenylnaphthyl residue of formula

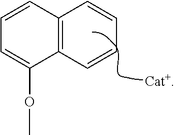

Herein, the Cat⁺ moiety denotes any suitable ammonium, pyridinium, phsophonium, phosphorinium, arsonium, sulfonium, and oxo sulfonium group. The Cat⁺ moiety may be bonded to any one of the three rings of the phenylnaphthyl residue.

An example is e.g. the substituted or unsubstituted phenylnaphthyl phosphonium residue of formula

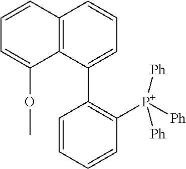 [SMILES: COc1cccc2cccc(-c3ccccc3[P+](c3ccccc3)(c3ccccc3)c3ccccc3)c12]

Another preferred group Cat⁺-Z—ArO— in which Z is an aryl linker is e.g. a substituted or unsubstituted binaphthyl residue of formula

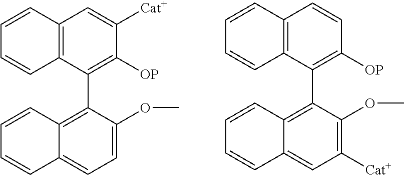

Herein, P denotes a protecting group, preferably a silyl group such as t-butyldimethylsilyl group, or an alkyl group such as C₁₋₄ alkyl.

Another preferred group Cat⁺-Z—ArO— in which Z is an aryl linker is e.g. a substituted or unsubstituted 5,6,7,8-tetrahydronaphthyl residue of formula

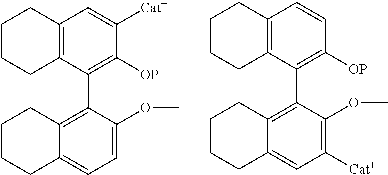

P is a protecting group as defined above.

The optional substituents of said phenylnaphthyl residue, binaphthyl residue or 5,6,7,8-tetrahydronaphthyl residue may be independently selected from C₁₋₁₀ alkyl, optionally substituted with halogen such as fluorine, C₁₋₁₀ alkoxy, nitro, cyano, phenyl, phenoxy, N(C₁₋₆ alkyl)₂, C(O)N(C₁₋₆ alkyl)₂, C(O)NH(C₁₋₆ alkyl), C(O)O—C₁₋₆ alkyl, halogen (F, Cl, Br, I) and two or more thereof.

Preferred binaphthyl residues and 5,6,7,8-tetrahydronaphthyl residues are

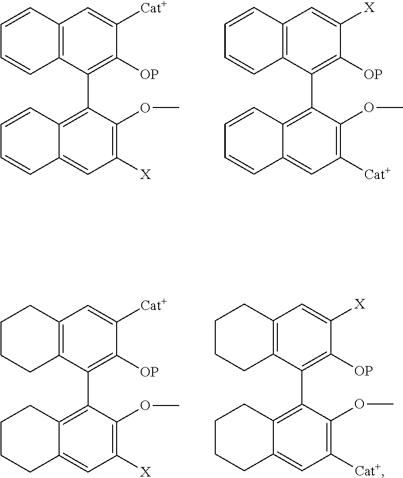

wherein X is F, Cl, Br or I, preferably F, Cl or Br.

Further according to the invention, R⁵ is alkyl or aryl, optionally substituted.

With reference to R⁵, the term “alkyl” denotes C₁₋₂₀ alky, and the term “aryl” denotes C₆₋₁₄ aryl.

A preferred alkyl residue R⁵ is 1-adamantyl or t-butyl.

A preferred aryl residue R⁵ is phenyl, optionally substituted.

Optional substituents are C₁₋₆ alkyl, optionally substituted with halogen such as fluorine, C₁₋₆alkoxy, halogen, nitro, cyano, phenyl, phenoxy, N(C₁₋₆alkyl)₂, C(O)N(C₁₋₆ alkyl)₂, C(O)NH(C₁₋₆ alkyl), C(O)O—C₁₋₆ alkyl, and two or more thereof.

Preferred residues R⁵ are 2,6-[(CH₃)₂CH]₂C₆H₃, 2,6-Cl₂C₆H₃, o-CF₃C₆H₄, o-t-C(CH₃)₃C₆H₄ and C₆F₅.

Further to the invention, the compound of formula I bears a non-nucleophilic anion. In a preferred embodiment, said said non-nucleophilic anion Y^{⊖} is selected from the group consisting of ClO₄^{⊖}, AsF₆^{⊖}, SbF₆^{⊖}, PF₆^{⊖}, CH₃SO₃^{⊖}, CF₃SO₃^{⊖}, p-CH₃C₆H₄SO₃^{⊖}, BF₄^{⊖}, B[3,6-(CF₃)₂C₆H₃]₄^{⊖}, B[C₆F₅]₄^{⊖} [=BF²⁰], Al[O-t-C(CH₃)(CF₃)₂]₄^{⊖}, and Al[O-t-C(CF₃)₃]₄^{⊖} [=Alpfb].

Preferred compounds of formula I are

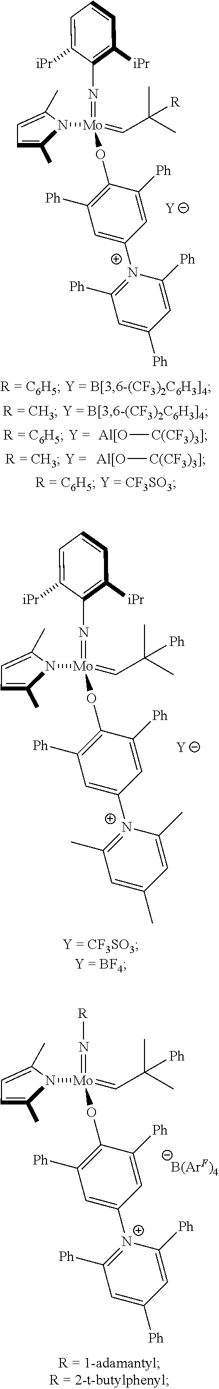

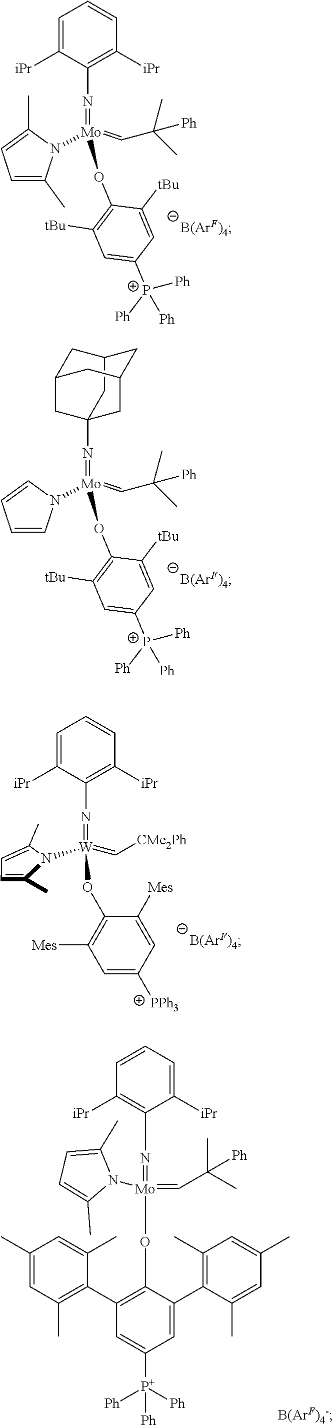

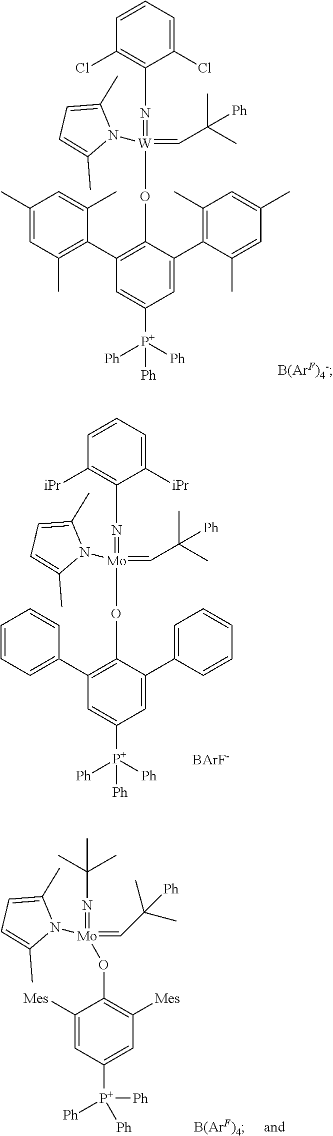

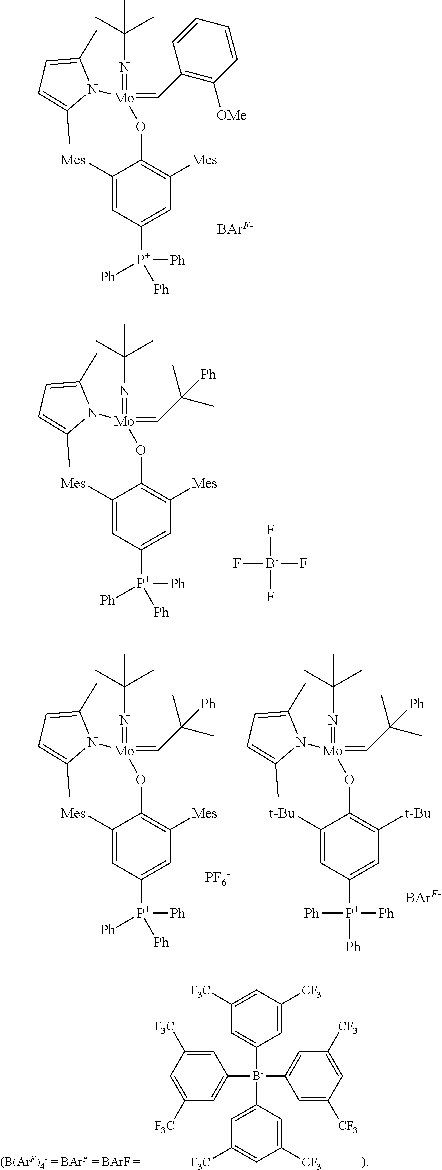

The compounds of formula I according to the invention—depending on the structure and the manufacturing method thereof—may also contain a neutral ligand stemming e.g. from the solvent in which the compound is prepared.

Suitable ligands are neutral ligands such as ethers such as THF or glycol ethers, nitriles such as acetonitrile, or pyridines.

The term “neutral ligand” as used herein does not encompass a nitrogen-containing carbene (NHC). Accordingly, the compounds of formula I are NHC-free.

The inventive compounds of formula I encompass compounds in which

X is O and R³ is a pyrrol-1-yl;

X is NR⁵ and R³ is a pyrrol-1-yl;

X is O and R³ is a halogen, preferably chlorine; and

X is NR⁵ and R³ is a halogen, preferably chlorine;

and wherein R⁴ may be broadly varied.

Accordingly, this variety of catalysts allows for a valuable tailor-made design which may be adapted to the specific olefins to be subjected to metathesis.

In a second aspect, the invention relates to a method of making a compound of formula I as defined in the first aspect, the method comprising step (A):

(A) reacting a compound of formula II

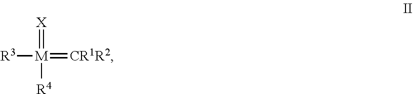

with a compound of formula III

[Cat⁺-Z—ArOH]Y^{⊖} III,
wherein M, X, R¹, R², R³, Cat⁺-Z—ArO and Y^{⊖} have the meaning as defined in the first aspect, and R⁴═R³,
to afford the compound of formula I.

This means that in the compound of formula III R⁴ is selected from a nitrogen-containing aromatic heterocycle being bound to M via said nitrogen; from halogen; and from triflate.

The compounds of formula II are known in the art and/or may be prepared by known methods.

The compounds of formula III are also known in the art and/or may be prepared by known methods. E.g., pyridinium N-phenol salts may be prepared by protonating a corresponding zwitterionic betaine dye with a respective acid. Zwitterionic dyes are known e.g. from Reichardt C., “Pyridinium N-phenolate betaine dyes as empirical indicators of solvent polarity: Some new findings”, Pure Appl. Chem. Vol. 76, No. 10, pp. 1903-1919, 2004; or Reichardt C. et al., “Solute/solvent interactions and their empitical determination by means of solvatochromic dyes”, Pure &Appl. Chem., Vol. 65, No 12, pp. 2593-2601, 1993.

In another approach, the anion of a pyridinium N-phenol salt may be exchanged by a non-nucleophile counterion.

Preferably, the compounds of formula II are reacted with a compound of formula III in a solvent such as THF or diethyl ether. Preferably, one equivalent of the compound of formula II is reacted with one equivalent of a compound of formula III. The products may be isolated according to known methods. Frequently, the compound of formula I precipitates and may be isolated by filtration. The yield of target compound typically is in the range of from 60 to 90%.

Exemplary reactions are depicted in Scheme 1 and Scheme 2 below where metal imido alkylidene compounds of formula II are reacted with a compound [Cat⁺-Z—ArOH]Y^{⊖} of formula III:

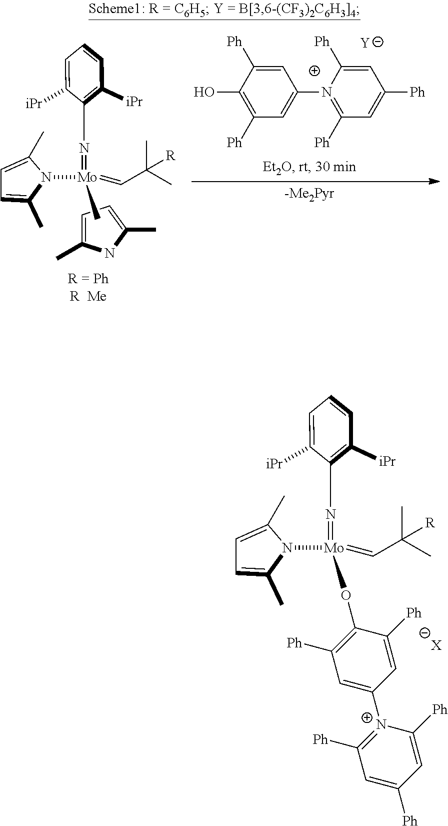

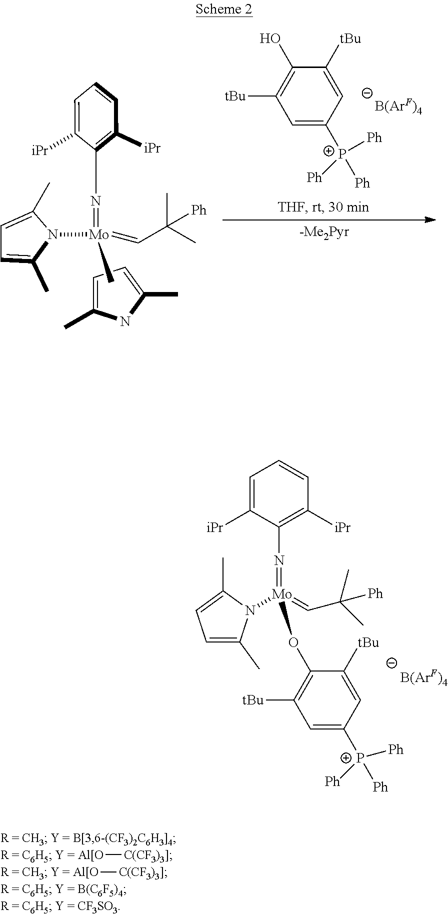

The reaction may also be performed in an analogous manner with a respective metal oxo alkylidene compound in place of a metal imido alkylidene compound.

In a third aspect, the invention relates to a composition comprising a compound as defined in the first aspect, and a solvent.

The term “solvent” as used herein encompasses any liquid which is suitable to dissolve or to disperse the compound of formula I without degradation.

In a preferred embodiment, the solvent is a solvent having a polarity being high enough to dissolve the compound.

In a preferred embodiment, the solvent is pyrrole, i.e. 1H pyrrole.

Further suitable solvents may be selected from the group consisting of acetonitrile, dimethyl formamide, dimethyl sulfoxide, hexamethyl phosphoramide, dimethyl acetamide, and sulfolane.

In a particularly preferred embodiment, the solvent is selected from an ionic liquid.

The term “ionic liquid” as used herein encompasses a salt in the liquid state. The term “ionic liquid” thus encompasses terms such as “liquid electrolyte”, “ionic melt”, “ionic fluid”, “fused salt”, “liquid salt” or “ionic glass”.

Preferably, the salt is liquid in a temperature range above −25° C., more preferably above −20° C. and most preferred above −15° C. Further particularly preferred, the salt is liquid at room temperature.

The inventors discovered that ionic liquids having a weakly coordinating anion are particularly useful solvents.

A weakly coordinating anion is tris(pentafluoroethyl)trifluorophosphate (FAP).

Another weakly coordinating anion is aluminum tetra[1,1,1,3,3,3-hexafluoro-2-propanolat] [Al(hfip)₄].

FAP comprising ionic liquids are preferred due to the high hydrophobicity of said anion.

Preferred ionic liquids are

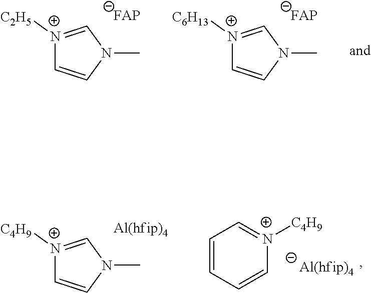

wherein the FAP-containing ionic liquids are preferred.

A further suitable ionic liquid is the known P66614⁺ cation with anions selected from FAP, NTf₂, PF₆⁻ and B(CN)₄⁻.

In a fourth aspect, the invention relates to a method of performing a metathesis reaction using the compound of formula I as defined in the first aspect or made according to a method as defined in the second aspect or using a composition as defined in the third aspect.

The term “metathesis reaction” encompasses any olefin metathesis reaction known in the art, preferably homo cross metathesis (HCM), cross metathesis (CM), ring-closing metathesis (RCM), ring opening metatheis (ROM), ring opening metathesis polymerization (ROMP), and acyclic diene metathesis (ADMET).

In one embodiment, the invention relates to a method of performing a metathesis reaction, comprising step (B):
(B) reacting a first olefin with a second olefin, wherein the first olefin is identical to or different from the second olefin, in the presence of a compound as defined in the first aspect, or in the presence of a composition as defined in the third aspect.

In a preferred embodiment, the metathesis reaction is performed in the presence of a composition as defined in the third aspect, and a further solvent. Preferably, the further solvent has a lower polarity than pyrrole or the ionic liquid such that said pyrrole or ionic liquid and the further solvent form two phases, i.e. a biphasic system.

In a preferred embodiment, the further solvent is selected from a hydrocarbon which is a liquid at room temperature. Suitable hydrocarbons are preferably C₅H₁₂ to C₁₀H₂₂ hydrocarbons.

In a preferred embodiment, the metathesis reaction is a ring closing reaction, i.e. the ring closing reaction of a compound having two terminal olefin groups wherein a cyclic compound is formed.

Accordingly, in one embodiment, the invention relates to a method of performing a ring closing metathesis reaction comprising
(a) the use of a compound as defined in the first aspect; or
(b) comprising the use of a composition as defined in the third aspect; or
(c) comprising the use of a composition as defined in the third aspect, and a further solvent, wherein the further solvent has a lower polarity than pyrrole, acetonitrile, dimethyl formamide, dimethyl sulfoxide, hexamethylphosphoramide, dimethylacetamide, and sulfolane or the ionic liquid such that said pyrrole, acetonitrile, dimethyl formamide, dimethyl sulfoxide, hexamethylphosphoramide, dimethylacetamide, and sulfolane or ionic liquid and the further solvent form two phases.

In a particularly preferred embodiment, the ring closing metathesis reaction is a macrocylisation of a compound having two terminal olefin groups in order to form a macrocycle.

The term “macrocycle” as used herein denotes a compound having at least 13 ring members.

In a particularly preferred embodiment, the macrocyclisation is performed such that it (c) comprises the use of a composition as defined in the third aspect, and a further solvent, wherein the further solvent has a lower polarity than pyrrole, acetonitrile, dimethyl formamide, dimethyl sulfoxide, hexamethylphosphoramide, dimethylacetamide, and sulfolane or the ionic liquid such that said pyrrole, acetonitrile, dimethyl formamide, dimethyl sulfoxide, hexamethylphosphoramide, dimethylacetamide, and sulfolane or ionic liquid and the further solvent form two phases. Preferably, said further solvent is a hydrocarbon.

It surprisingly has been discovered that (a) using a compound as defined in the first aspect or (b) a composition as defined in the third aspect or (c) a composition and a further solvent as defined herein in the fourth aspect advantageously may allow reducing the tendency known in macrocyclisation reactions that the starting material to be subjected to cyclisation reacts intermolecularly instead of intramolecularly.

Table 1 shows the application of

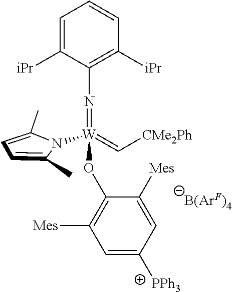

in standard CM reactions:

TABLE 1
Productivities expressed in TONs in RCM and HCM reactions using
different solvent systems; values in brackets: E/Z

| Substrate/Solvent | | | |
|---|---|---|---|
| System | toluene^{[a]} | pyrrole/heptane^{[b]} | IL1/heptane^{[c]} |
| 1,7-octadiene | 515 | 840 | 780 |
| 1-hexene | 250 | 120 | 170 |
| allyl benzene | 50 (97/3) | 60 (99/1) | 30 (87/13) |
| allyl trimethylsilane | 250 | 280 | 210 |
| 1-dodecene | 150 | 200 | 230 |
| 1-octene | 220 (2/98) | 240 (3/97) | 280 (6/94) |
| allyl phenyl sulfide | 120 (5/95) | 150 (5/95) | 115 (7/93) |

^{[a]}25° C., dodecane as internal standard, 6 h, catalyst:substrate = 1:1000.
^{[b]}pyrrole:heptane (2:3), 25° C., dodecane as internal standard, 6 h, catalyst:substrate = 1:1000.
^{[c]}IL1:heptane (1:3), 25° C., mesitylene as internal standard, 6 h, catalyst:substrate = 1:1000.

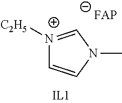

TONs obtained in toluene, pyrrole/heptane or IL1/heptane, respectively, were comparable. Notably, the catalyst showed high Z-selectivity up to 98% in the HM of 1-octene and allyl phenyl sulphide.

The metal content of the nonpolar phase was determined by inductively coupled plasma-optical emission spectroscopy (ICP-OES) measurements. The reactions of the catalyst with 1,7-octadiene and 1-hexene did not show any migration of tungsten into the heptane phase with IL1 as well as pyrrole, i.e. the metal content was below the limit of detection (<2 ppm).

Table 2 shows the application of

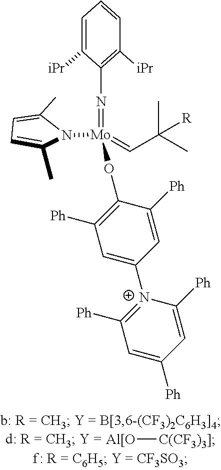

in standard RCM and HCM:

TABLE 2
Productivities expressed in TONs in RCM and HCM reactions using
chlorobenzene and ionic liquid IL1/heptane as solvents

| Substrate | b^{[a]} | b biphasic^{[b]} | d^{[a]} | ′d biphasic^{[b]} | f^{[a]} | ′f biphasic^{[b]} |
|---|---|---|---|---|---|---|
| RCM | | | | | | |
| diethyl diallylmalonate | 1600 | 880 | 1500 | 900 | 815 | 850 |
| diallylmalodinitrile | 710 | 510 | 810 | 750 | 550 | 560 |
| N,N-diallyltosylamine | 4600 | 4100 | 5500 | 5100 | 4200 | 4300 |
| diallyl sulfide | 4700 | 3900 | 5100 | 4800 | 4150 | 4000 |
| 1,7-octadiene | 10000 | 10000 | 10000 | 10000 | 10000 | 10000 |
| HM | | | | | | |
| methyl oleate | 800 | 710 | 960 | 500 | 590 | 630 |
| 1-octene | 2900 | 2750 | 3300 | 3200 | 2760 | 2900 |
| allylbenzene | 850 | 810 | 870 | 860 | 580 | 620 |
| 9-DAME | 740 | 690 | 750 | 710 | 550 | 510 |
| allyl benzyl ether | 1330 | 1260 | 1350 | 1300 | 1100 | 990 |
| 5-hexenyl acetate | 420 | 440 | 580 | 600 | 550 | 530 |
| N-allyl-N-phenylamine | 10 | 15 | 15 | 20 | 25 | 15 |

^{[a]}chlorobenzene, 25° C., dodecane as internal standard, 8 h, catalyst:substrate = 1:10,000
^{[b]}IL1/heptane (1:3), 25° C., mesitylene as internal standard, 8 h

Homogeneous and biphasic reaction setups produced in most cases comparable TONs. Notably, TONs in the thousands were reached with substrates containing functional groups such as N,N-diallyltosylamine or diallyl sulphide. Catalyst b was chosen to determine the maximum TON for 1,7-octadiene. With a loading of 200,000 equivalents of octadiene with respect to catalyst, a TON of 150,000 can be obtained in solution. That highlights the potential of these catalysts for the conversion of simple olefins. Under biphasic conditions, using solely ionic liquid IL1 and pure substrate, the maximum TON was 66,000.

Advantageously, the catalysts may be reused as shown for catalyst d in IL1. A solution of catalyst d in IL1 was stored in a freezer for 2 to 3 days. No loss of activity was observed when reused in metathesis.

In summary, the compounds of formula I have been isolated and successfully applied to a biphasic metathesis reaction. Reactions in ionic liquids produce similar results for a number of substrates when compared to a homogeneous reaction with common solvents such as chlorobenzene or toluene. The products are obtained in a virtually metal-free form (<2 ppm) as evidenced by ICP-OES measurements. Furthermore, the new ionic catalysts have good stability both under storage conditions and reaction conditions.

Further preferred catalysts are

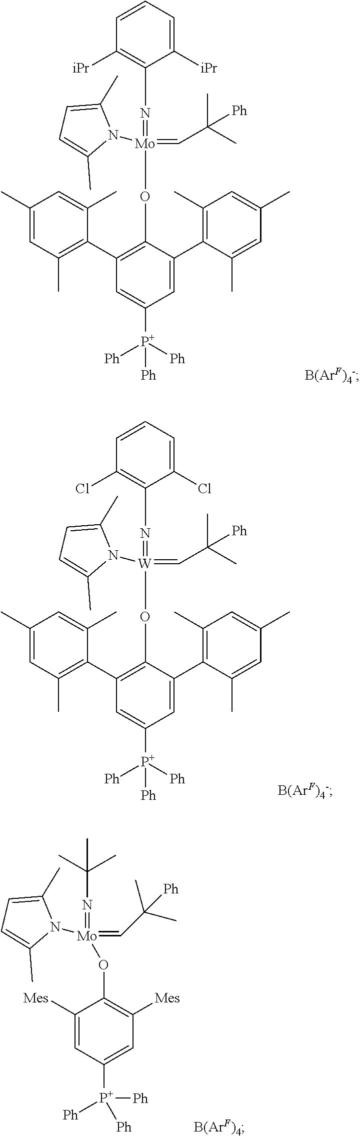

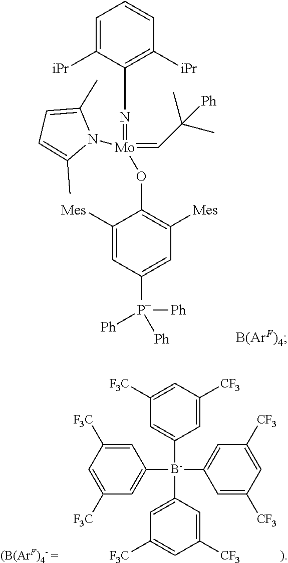

In a fifth aspect, the invention may be extended to compounds of formula IV

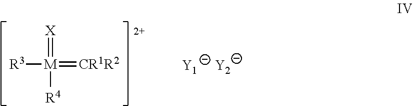

wherein:
M is selected from Mo or W;
X is selected from O or NR⁵;
R¹ and R² are independently selected from H, C₁₋₆ alkyl, and aryl; C₁₋₆ alkyl and aryl optionally being substituted with one or more of C₁₋₆alkyl, C₁₋₆ alkoxy, and O—C₆H₅;
R³ and R⁴ are independently from each other an aryl oxy group being bound to M via said oxygen of said aryl oxy group; wherein said aryl group of said aryl oxy group is bound to a group Cat such to form a cationic ligand Cat⁺-Z—ArO—, wherein Z is either a covalent bond or a linker;
R⁵ is alkyl or aryl, optionally substituted; and
Y₁^{⊖} and Y₂^{⊖} are triflate, respectively.

M, X, R¹, R², R⁵ and Cat⁺-Z—ArO— have the same meaning as defined in the first aspect.

In a preferred embodiment, R³ and Ware identical, i.e. R³═R⁴.

In a sixth aspect, the invention relates to a method of making a compound of formula IV as defined in the fifth aspect, the method comprising step (A):

(A) reacting a compound of formula V

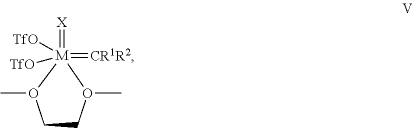

wherein X, R¹ and R² have the meaning as defined in the fifth aspect, and TfO is triflate,
with a compound of formula VI

Cat⁺-Z—ArO^{⊖} VI
wherein Cat⁺-Z—ArO has the meaning as defined in the fifth aspect and the compound of formula VI is a zwitterion,
to afford the compound of formula IV.

Preferred zwitterions are the ions specified in the Reichardt-references mentioned above:

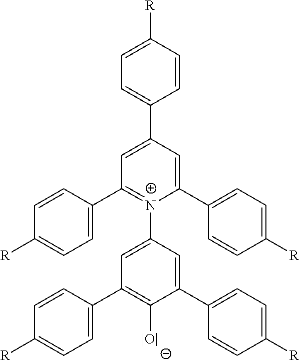

wherein R is H, C(CH₃)₃, C₆H₅, CF₃ or C₆F₁₃;

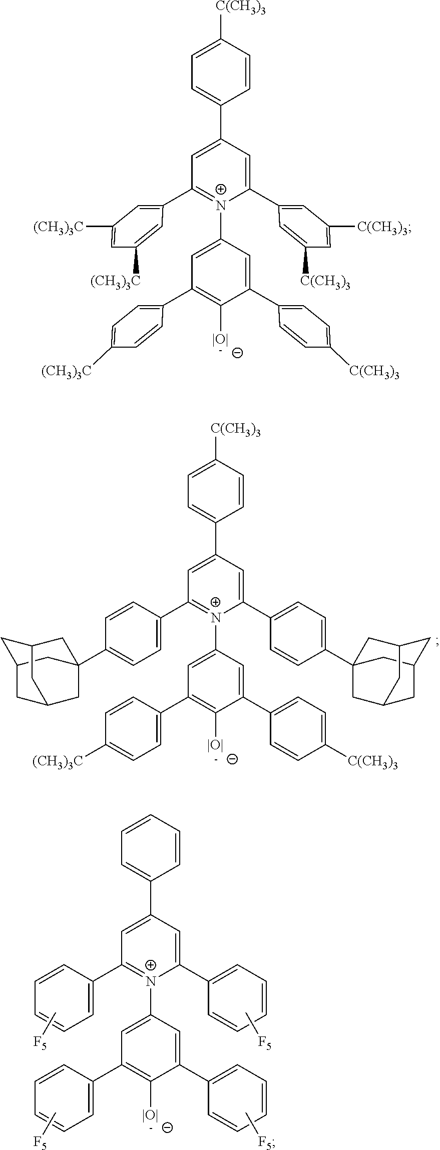

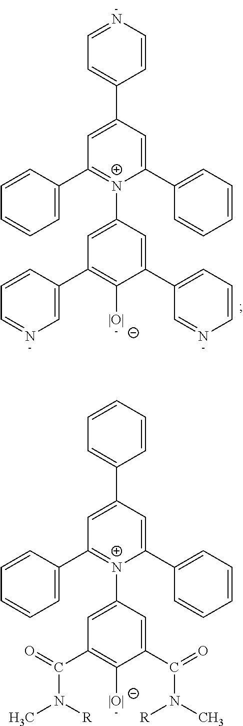

wherein R is H or CH₃.

A further preferred zwitterion is a zwitterion Cat⁺-Z—ArOe in which Z is a linker such as

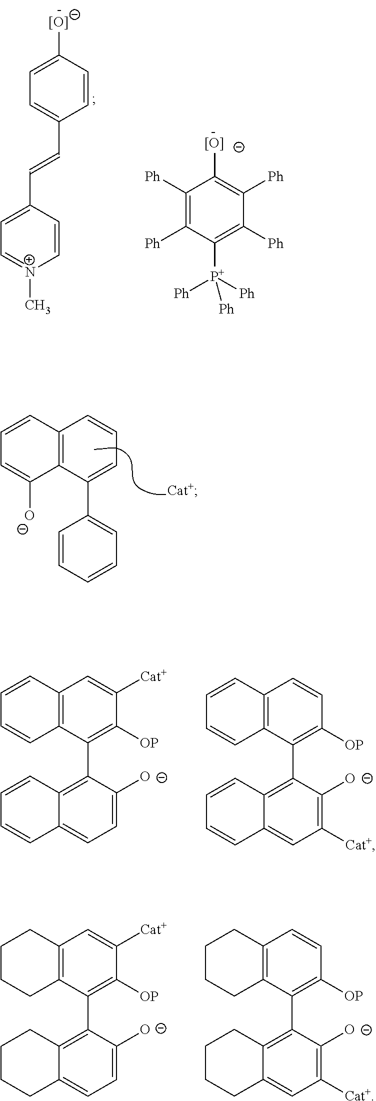

The reaction requires that two equivalents of the compound of formula VI are reacted with one equivalent of the compound of formula V.

In a seventh aspect, the invention relates to a composition comprising a compound of formula IV as defined in the fifth aspect, and a solvent.

The same definitions regarding the solvent as in the third aspect apply.

In an eighth aspect, the invention relates to a method of performing a metathesis reaction using the compound of formula IV as defined in the fifth aspect. The same definitions regarding the method as in the fourth aspect apply.

Table 3 shows the application of compound IVa (X=2,6-iPr₂—C₆H₃—N; R¹, R²=H, CMe₂Ph), IVb (X=2-CF₃—C₆H₄—N; R¹, R²=H, CMe₂Ph); IVc (X=2,6-Me₂-C₆H₃—N; R¹, R²=H, CMe₂Ph); IVd (X=2,6-Cl₂—C₆H₃—N; R¹, R²=H, CMe₃); and IVe (X=adamant-1-yl-N; R¹, R²=H, CMe₂Ph); and R³═R⁴=

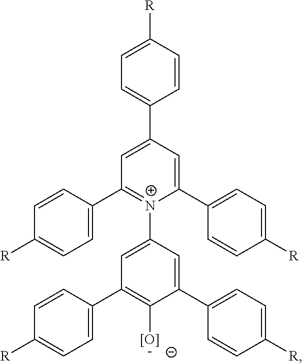

respectively, in standard CM reactions:

TABLE 3
Productivities expressed in TONs in RCM and HCM:

| | | | IVb | | | IVd | |
|---|---|---|---|---|---|---|---|
| Substrate | IVa^{[a]} | IVb^{[a]} | bisphasic^{[b]} | IVc^{[a]} | IVd^{[a]} | biphasic^{[b]} | IVe^{[a]} |
| RCM | | | | | | | |
| diethyl diallylmalonate | 5 | 55 | 50 | 9 | 40 | 38 | 8 |
| diallylmalodinitrile | 3 | 43 | 40 | 6 | 33 | 35 | 4 |
| N,N-diallyltosylamine | 7 | 79 | 85 | 9 | 65 | 71 | 5 |
| diallyl sulfide | 3 | 57 | 55 | 5 | 43 | 35 | 4 |
| 1,7-octadiene | 20 | 360 | 405 | 27 | 350 | 406 | 23 |
| HM | | | | | | | |
| methyl oleate | 0 | 26 | 20 | 0 | 15 | 13 | 0 |
| 1-octene | 8 | 39 | 44 | 11 | 45 | 40 | 7 |
| allylbenzene | 3 | 29 | 20 | 4 | 22 | 16 | 4 |
| 9-DAME | 2 | 25 | 21 | 3 | 20 | 13 | 2 |
| allyl benzyl ether | 3 | 39 | 46 | 4 | 21 | 28 | 3 |
| 5-hexenyl acetate | 0 | 27 | 22 | 1 | 25 | 17 | 0 |

^{[a]}chlorobenzene, 25° C., dodecane as internal standard, 8 h, catalyst:substrate = 1:1000.
^{[b]}IL1:heptane = 1:3, 25° C., mesitylene as internal standard, 8 h, catalyst:substrate = 1:1000, 9-DAME = 9-decenoic acid methyl ester.

In a ninth aspect, the invention relates to a compound of formula VII

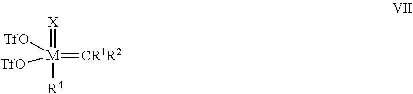

wherein
M is selected from Mo or W;
X is selected from 0 or NR⁵;
R¹ and R² are independently selected from H, C₁₋₆ alkyl, and aryl; C₁₋₆ alkyl and aryl optionally being substituted with one or more of C₁₋₆alkyl, C₁₋₆ alkoxy, and O—C₆H₅;

R⁴ is an aryl oxy group being bound to M via said oxygen of said aryl oxy group; wherein said aryl group of said aryl oxy group is bound to a group Cat such to form a cationic ligand Cat⁺-Z—ArO—, wherein Z is either a covalent bond or a linker;

TfO has the meaning of CF₃SO₂; and wherein the positive charge of the cationic ligand is compensated by a negative charge in the compound.

X, R¹, R², R⁵ and Cat⁺-Z—ArO— have the same meaning as defined in the first aspect.

NMR spectroscopic investigations indicate that the metal center M is anionic.

According to a tenth aspect, the invention relates to a method of making a compound of formula VII, wherein a compound of formula V is reacted with a compound of formula VI as defined in the sixth aspect.

The reaction requires that one equivalent of the compound of formula VI is reacted with one equivalent of the compound of formula V.

In an eleventh aspect, the invention relates to a composition comprising a compound as defined the tenth aspect, and a solvent.

The solvent is a solvent as defined in the third aspect.

In a twelfth aspect, the invention relates to a method of performing a metathesis reaction using the compound of formula VII as defined in the ninth aspect.

The same definitions regarding the method as in the fourth aspect apply.

### Examples

General Information

All reactions were performed under the exclusion of air and moisture by standard Schlenk techniques unless otherwise noted. Reactions involving metal complexes were performed in an N₂ filled glove box (MBraun Labmaster 130). Glassware was either stored at 120° C. overnight and cooled in an evacuated antechamber or dried at 500° C. under high vacuum (0.01 mbar).

¹H and ¹³C NMR spectra were recorded on a Bruker Avance III 400 spectrometer at 400 and 100 MHz, respectively. Chemical shifts are reported in ppm from tetramethylsilane with the solvent resonance resulting from residual solvent protons (CDCl₃: 7.26 ppm, C₆D₆ 7.16 ppm, CD₂Cl₂ 5.13 ppm) as reference. Data are reported as follows: chemical shift, multiplicity (s=singlet, d=doublet, t=triplet, q=quartet, quint=quintet, sept=septet, br=broad, m=multiplet), integration and coupling constants (Hz).

Elemental analyses were carried out at the Institute of Inorganic Chemistry, University of Stuttgart, Germany.

CH₂Cl₂, THF, diethyl ether, toluene and pentane were dried by using an MBraun SPS-800 solvent purification system with alumina drying columns and stored over 4 Å Linde type molecular sieves (toluene, CH₂Cl₂, Et₂O, pentane). THF was additionally distilled from Na prior to use. Deuterated solvents were filtered over activated alumina and stored over 4 Å Linde type molecular sieves inside the glove box. All reagents were purchased from commercial sources (ABCR, TCI, ACROS-Organics, Sigma-Aldrich, Alfa Aesar) and used as received unless otherwise noted.

Microwave-Assisted Digestion

Microwave program for ICP-OES samples.

| t [min] | Power [W] | T [° C.] |
|---|---|---|
| 20 | 0 | r.t. |
| 10 | 600 | r.t. to 160° C. |
| 60 | 600 | 160° C. |
| 30 | 0 | 160° C. to r.t. |

General Conditions for Homogeneous Reactions

Substrate (1000 equivalents with respect to the catalyst) was dissolved in 0.3 mL of dry DCM. Subsequently, a stock solution of the catalyst (0.05M in DCM) was added. The reaction mixture was stirred for 20 h at RT (closed vial). A sample for GC-MS was withdrawn to determine conversion and E/Z ratio.

General Conditions for Biphasic Reactions in Pyrrole/Heptane

Substrate (1000 equivalents with respect to the catalyst) was dissolved in 0.3 mL of heptane. Subsequently, a stock solution of the catalyst in pyrrole (0.2 mL, 1 mg mL⁻¹) was added. The reaction mixture was stirred for 20 h at RT (closed vial). The mixture was homogenized by adding DCM. A sample for GC-MS was withdrawn to determine the conversion and E/Z ratio.

General conditions for biphasic reactions in IL1/heptane

Catalyst was weighted as a solid (1-2 mg) followed by addition of 0.1 mL ionic liquid. Subsequently, the substrate (1000 equivalents with respect to the catalyst) was dissolved in 0.3 mL of heptane and transferred to the catalyst solution. The reaction mixture was heavily stirred for 20 h at RT (closed vial). The upper heptane layer was collected by decantation and analyzed by GC-MS to determine the conversion and E/Z ratio. Then another batch of substrate in heptane was added to the catalyst and the process was repeated.

Syntheses of Ligands

1-(3,5-Diphenyl-4-hydroxyphenyl)-2,4,6-triphenylpyridin-1-ium tetrafluoroborate [B. P. Johnson, B. Gabrielsen, M. Matulenko, J. G. Dorsey, C. Reichardt, Anal. Lett. 1986, 19, 939-962].

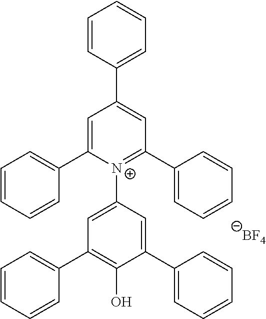

The commercially available betaine

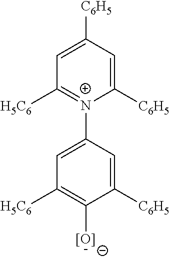

(200 mg, 0.36 mmol) was suspended in 20 mL of water. Under heavy stirring an aqueous solution of HBF₄ (48%, 1 mL, excess) was added drop wise. The dark green betaine became slowly colorless. After 2 h a pale-yellow solid was filtered off and washed with diethyl ether (230 mg, 99%).

¹H-NMR (400 MHz, DMSO-d₆): δ=7.14 (d, J=7.07 Hz, 4H), 7.33 (m, 4H), 7.39 (m, 4H), 7.47 (m, 6H), 7.56 (m, 4H), 7.68 (m, 3H), 8.37 (d, J=8.36 Hz, 2H), 8.70 (s, 2H), 8.76 (s, 1H) ppm; ¹³C-NMR (100 MHz, DMSO-d₆): δ=124.9, 127.5, 128.2, 128.3, 128.8, 129.1, 129.7, 129.8, 129.9, 130.4, 131.6, 132.5, 133.4, 133.5, 136.9, 150.7, 155.3, 156.6 ppm; ¹⁹F-NMR (375 MHz, DMSO-d₆): δ=−148.25, 148.30 ppm. IR (ATR): V̅=3517 (vw), 3058 (vw), 1623 (s), 1555 (m), 1419 (m), 1231 (m), 1048 (vs, br), 760 (s), 694 (vs).

### 1-(3,5-Diphenyl-4-hydroxyphenyl)-2,4,6-triphenylpyridin-1-ium triflate

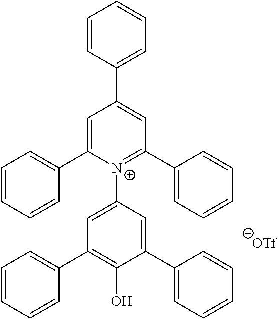 [SMILES: Oc1c(-c2ccccc2)cc(-[n+]2c(-c3ccccc3)cc(-c3ccccc3)cc2-c2ccccc2)cc1-c1ccccc1]

1-(3,5-Diphenyl-4-hydroxyphenyl)-2,4,6-triphenylpyridin-1-ium chloride (414 mg, 0.704 mmol) was dissolved in 15 mL CH₂Cl₂. To the yellow solution AgOTf (199 mg, 0.774 mmol, 1.1 equiv.) was added as a solid. Under the exclusion of light the suspension was stirred for 30 min at room temperature. After filtration over celite the solvent was removed in vacuo. The yellow oily residue was dissolved in 1 mL CH₂Cl₂ and subjected to flash chromatography using CH₂Cl₂ as eluent. After collecting the yellow band and removing the solvent in vacuo the residue was dissolved in 5 mL trichloroethylene. The pale-yellow product precipitated after a few minutes. It was filtered off and washed with 2 mL trichloroethylene (426 mg, 86%).

¹H-NMR (400 MHz, CDCl₃): δ=8.10, 7.88, 7.55, 7.40, 7.14, 5.55 ppm; ¹³C-NMR (101 MHz, CDCl₃): 5=157.7, 157.1, 150.0, 135.2, 134.7, 133.4, 132.2, 131.8, 130.4, 130.0, 129.8, 129.6, 129.3, 129.2, 129.1, 128.7, 128.6, 128.6, 126.5, 120.9 (q, J=321.17 Hz, OTf) ppm; ¹⁹F-NMR (376 MHz, CDCl₃): 5=−78.03 ppm. Elemental analysis (%) calcd. for C₄₂H₃₀F₃NO₄S: C, 71.89; H, 4.31; N, 2.00. Found: C, 71.82; H, 4.468; N, 2.16.

### 1-(3,5-Diphenyl-4-hydroxyphenyl)-2,4,6-triphenylpyridin-1-ium B[C₆F₅]₄^{⊖}

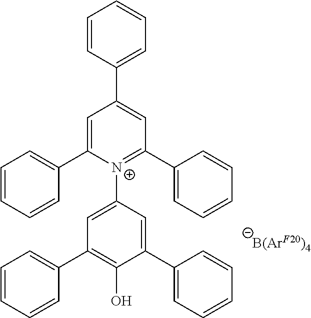 [SMILES: Oc1c(-c2ccccc2)cc(-[n+]2c(-c3ccccc3)cc(-c3ccccc3)cc2-c2ccccc2)cc1-c1ccccc1]

1-(3,5-Diphenyl-4-hydroxyphenyl)-2,4,6-triphenylpyridin-1-ium tetrafluoro-borate (225 mg, 0.352 mmol) was dissolved in 10 mL CH₂Cl₂. To the yellow solution KB(C₆F₅)₄ (253 mg, 0.352 mmol, 1 equiv.) was added as a solid. The suspension was stirred overnight at room temperature. A white precipitate was filtered through a pad of celite. After removing the solvent in vacuo, the residue was taken up in 5 mL of CH₂Cl₂ and filtered over a pad of silica. CH₂Cl₂ was removed in vacuo. The product was obtained as yellow foam. After stirring in 3 mL diethyl ether an off white solid precipitated and was filtered off. (390 mg, 85%).

¹H-NMR (400 MHz, CDCl₃): δ=8.24, 7.82, 7.68, 7.59, 7.53, 7.45, 7.39, 7.31, 7.04, 6.85, 5.63 ppm; ¹³C-NMR (101 MHz, CDCl₃): δ=158.3, 157.5, 150.7, 149.4, 147.0, 139.4, 137.5, 136.9, 135.1, 134.5, 133.7, 132.9, 132.3, 131.4, 130.7, 130.4, 130.2, 129.4, 129.3, 129.1, 128.9, 128.8, 128.1, 125.7 ppm; ¹⁹F-NMR (376 MHz, CDCl₃): δ=−132.50, −162.94, −166.81 ppm. Elemental analysis (%) calcd. for C₆₉H₄₀BF₂₀NO₂: C, 63.46; H, 3.09; N, 1.07. Found: C, 63.22; H, 2.794; N, 1.34.

### 2,6-Di(2,4,6-(trimethyl)phenyl)-4-bromophenol

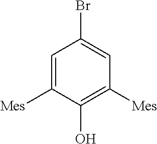

2,6-Di(2,4,6-(trimethyl)phenyl)phenol (492 mg, 1.5 mmol) was dissolved in glacial acetic acid (20 mL). A solution of bromine (72 μL, 226 mg, 1.4 mmol) in glacial acetic acid (5 mL) was slowly dropped to this solution under vigorous stirring. After two hours, demineralized water was added and the resulting colourless residue was filtered off and washed with water. Then the solid was dissolved in diethyl ether and the organic phase was washed with water and brine and was subsequently dried over sodium sulfate. The solvent was evaporated. According to GC-MS the crude product contained approximately 2% educt and a double brominated species. The crude product was used without further purification

### (3,5-Di-(2,4,6-(trimethyl)phenyl)-4-hydroxyphenyl)triphenyl-phosphonium bromide

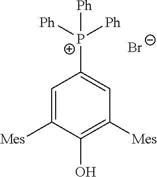

Bromophenol from the previous step (822 mg, 0.2 mmol), triphenylphosphine (789.5 mg, 0.3 mmol), tris(dibenzylideneacetone) dipalladium (55 mg, 3 mol %) were suspended in ethylene glycol (2 mL, dry) and heated to 130° C. After 20 h the solvent was removed by distillation and the resulting residue was purified by column chromatography (silica, methanol/dichloromethane, 1/10). The product was isolated in 60% yield as a colorless foam.

¹H-NMR (CDCl₃): δ=7.93-7.89 (m, 3H), 7.82-7.77 (m, 6H), 7.68-7.63 (m, 6H), 7.26 (s, 1H), 7.23 (s, 1H), 6.99 (s, 4H), 2.31 (s, 6H), 2.03 (s, 12H) ppm; ¹³C-NMR (CD₃CN): δ=158.6 (d), 139.1, 137.6, 137.4 (d), 136.2 (d), 135.5 (d), 132.9, 131.5 (d), 131.3 (d), 129.3, 119.7 (d), 108.3 (d), 21.2, 20.6 ppm. HRMS-ESI calcd. for C₄₂H₄₀OP⁺: 591.2811. Found 591.2819.

### (3,5-Di-(2,4,6-(trimethyl)phenyl)-4-hydroxyphenyl)triphenyl-phosphonium B[3,6-(CF₃)₂C₆H₃]₄^{⊖}

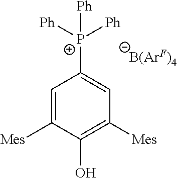

The bromide salt from the previous step (444 mg, 0.6 mmol) was dissolved in dichloromethane and NaBAr^{F} (584 mg, 0.6 mmol) in a mixture of dichloromethane and diethyl ether was added. After 14 h the resulting suspension was filtered over silica and the solvent was removed under reduced pressure. The product was isolated in 78% yield as a colorless foam.

¹H-NMR (400 MHz. C₆D₆): δ=7.71-7.66 (m, 3H), 7.56-7.51 (m, 14H), 7.47-7.41 (m, 6H), 7.39 (br s, 4H), 7.14 (s, 1H), 7.11 (s, 1H), 6.83 (s, 4H), 5.40 (s, 1H), 2.13 (s, 6H), 1.85 (s, 12H) ppm; ¹⁹F-NMR (375 MHz, C₆D₆): δ=−62.88 ppm; ¹³C-NMR (101 MHz, CD₃CN): δ=162.6 (q), 158.5 (d), 139.2, 137.7, 137.4 (d), 136.2 (d), 135.7, 135.5 (d), 132.7, 131.2 (d), 130.5-129.7 (m), 129.3, 126.8, 124.1, 120.2, 119.3, 108.4 (d), 21.1, 20.5 ppm. Elemental analysis (%) calcd. for C₇₄H₅₂BF₂₄OP: C, 61.09; H, 4.03. Found: C, 61.47; H, 4.03.

### Example 1: Synthesis of

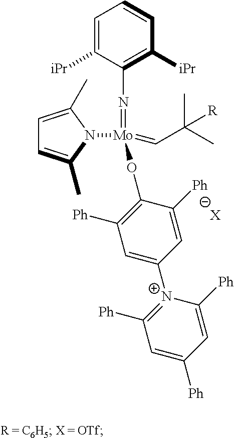

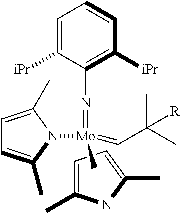

(55.6 mg, 0.094 mmol) was dissolved in 2 mL diethyl ether.

The solution was cooled to −35° C. 1-(3,5-Diphenyl-4-hydroxyphenyl)-2,4,6-triphenylpyridin-1-ium triflate (65.3 mg, 0.094 mmol) was added as a solid. The color of the mixture changed to red-orange. The mixture was stirred for another 30 min. An orange powder formed, which was filtered off and washed with 5 mL diethyl ether.

¹H-NMR (400 MHz, CD₂Cl₂): δ=11.07 (s, ¹J_{CH}=119.6 Hz, 1H, Mo═CH), 8.20 (s, 2H, Ar), 8.02-7.89 (m, 2H, Ar), 7.71-7.59 (m, 4H, Ar), 7.59-7.48 (m, 4, 7.46-7.30 (m, 7H, Ar), 7.28-7.19 (m, 11H, Ar), 7.19-7.10 (m, 6H, Ar), 7.19-7.02 (m, 1H, Ar), 7.02-6.88 (m, 4H, Ar) 5.73 (s, 2H, pyr), 2.71 (sept, ³J_{HH}=6.3 Hz, 2H, iPr), 1.90 (s, br, 6H, pyr-Me), 1.32 (s, 3H, CMe₂Ph), 1.26 (s, 3H, CMe₂Ph), 0.76 (d, ³J_{HH}=5.6 Hz, 6H, iPr-Me), 0.65 (d, ³J_{HH}=6.2 Hz, 6H, iPr-Me) ppm; ¹⁹F-NMR (376 MHz, CD₂Cl₂): δ=−78.65 (s, 6F, OTf) ppm; ¹³C-NMR (101 MHz, CD₂Cl₂): δ=293.5 (Mo═CH), 160.5 (Ar), 157.9 (Ar), 157.6 (Ar), 154.1 (Ar), 148.1 (Ar), 147.9 (Ar), 137.8 (Ar), 134.8 (Ar), 134.5 (Ar), 133.8 (Ar), 133.7 (Ar), 133.0 (Ar), 132.3 (Ar), 130.9 (Ar), 130.5 (Ar), 130.4 (Ar), 130.3 (Ar), 129.6 (Ar), 129.3 (Ar), 129.1 (Ar), 128.9 (Ar), 128.7 (Ar), 128.5 (Ar), 126.9 (Ar), 126.7 (Ar), 126.4 (Ar), 123.3 (Ar), 121.5 (q, ¹J_{CF}=321.4 Hz, CF₃), 109.2 (pyr), 56.5 (CMe₂Ph), 31.8 (CMe₂Ph), 30.3 (CMe₂Ph), 28.7 (iPr-CH), 23.8 (iPr-Me), 23.3 (iPr-Me), 16.9 (pyr-Me) ppm. Elemental analysis (%) calcd. for C₇₀H₆₆F₃MoN₃O₄S: C, 70.16; H, 5.55; N, 3.51. Found: C, 70.00; H, 5.714; N, 3.57.

### Example 2: Synthesis of

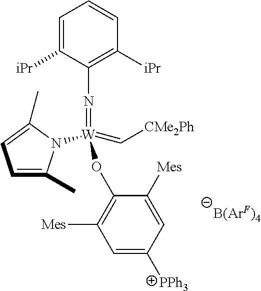

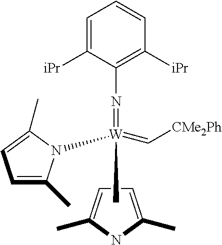

(50 mg, 0.01 mmol) was dissolved in benzene and 3,5-Di-(2,4,6-(trimethyl)phenyl)-4-hydroxyphenyl)triphenylphosphonium B(Ar^{F})₄⁻ (99 mg, 0.10 mmol) was added as a solid. The suspension was heated to 70° C. for 12 h. The solvent was evaporated from the yellow solution and the resulting residue was washed with a mixture of pentane and diethyl ether to afford the target compound in quantitative yield as yellow foam.

¹H-NMR (300 MHz, C₆D₆): δ=8.70 (s, 1H), 8.38 (br s, 8H, o-H, B(Ar^{F})₄), 7.57 (s, 4H, p-H, B(Ar^{F})₄), 7.26-7.24 (m, 2H), 7.17-7.13 (m, 2H), 7.07-6.91 (m, 18H), 6.83-6.78 (m, 6H), 6.73-6.70 (m, 5H), 6.01 (s, 1H), 5.79 (s, 1H), 3.10-2.99 (m*, 2H, H₃C—CH—CH₃, iPr), 2.27 (s, 3H), 2.09 (s, 6H, H₃C, HMTO), 1.94 (s, 6H, H₃C, HMTO), 1.84 (s, 6H, H₃C, HMTO), 1.77 (s, 3H), 1.55 (s, 3H), 1.32 (s, 3H), 1.12 (s, 3H, H₃C, iPr)**, 1.06 (s, 3H, H₃C, iPr)**, 0.95 (s, 3H, H₃C, iPr)**, 0.66 (s, 3H, H₃C, iPr)** ppm; ¹⁹F-NMR (377 MHz, C₆D₆): δ=−62.19 (B(Ar^{F})₄) ppm; ¹³C-NMR (101 MHz, C₆D₆): δ=267.2 (W═CH), 165.4 (d, ⁴J_{CP}=3.3 Hz, ipso-C, OHMT), 162.8 (q, ¹J_{CB}=50 Hz, B(Ar^{F})₄), 151.8, 151.1, 149.1, 144.7, 139.0, 137.5, 137.3, 136.2 (d, ²J_{CP}=19.1 Hz, m-C, OHMT), 135.7 (d, ³J_{CP}=14.2 Hz, o-C, OHMT), 135.42 (B(Ar^{F})₄), 133.7 (d, ³J_{CP}=10.5 Hz, m-C, PPh₃), 132.7, 132.7, 130.3 (d, ²J_{cp}=12.5 Hz, o-C, PPh₃), 130.1 (m), 129.8 (m), 129.4, 129.3, 128.5, 126.6, 126.5, 126.4, 123.9, 123.1 (d, ¹J_{CP}=71.1 Hz, p-C, OHMT), 121.1, 118.3, 118.1 (m), 117.4, 111.3 (d, ¹J_{CP}=39.5 Hz, ipso-C, PPh₃), 109.1, 108.2, 106.5, 54.47, 33.6, 30.9, 28.8, 28.1, 23.5, 23.1, 21.1, 20.8, 19.0, 15.2 ppm. *Expected: two septets, not resolved. **Expected: doublets, not resolved. Elemental analysis (%) calcd. for C₁ₒ₂H₈₈BF₂₄N₂OPW: C, 60.07; H, 4.35; N, 1.37. Found: C, 59.69; H, 4.687; N, 1.72.

### Example 3: Synthesis of

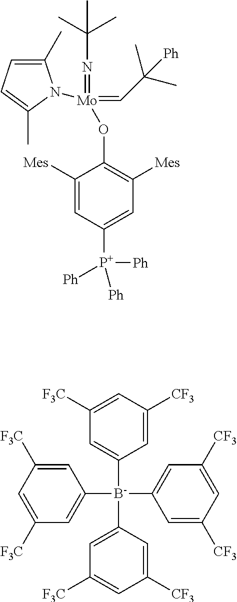

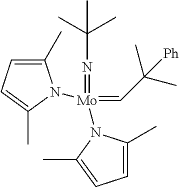

(24 mg, 0.05 mmol) was dissolved in benzene (3 mL) and

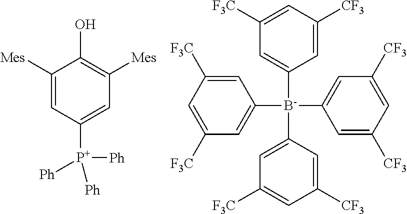

(73 mg, 0.10 mmol) was added as a solid. The suspension was stirred at room temperature for 12 h. The solvent was evaporated from the yellow solution and the resulting residue was taken in toluene (3 mL) which was subsequently evaporated. This operation was repeated 2 times to afford the target compound in 84% yield (78 mg) as a dark orange foam.

¹H-NMR (300 MHz, C₆D₆): δ=0.87 (s, 9H, ^{t}Bu), 1.37 (s br, 3H, CH₃ neophilydene), 1.67 (s, 3H, CH₃ neophilydene), 1.80 (s, 6H, CH₃ Mes ortho), 1.87 (s, 6H, CH₃ Mes ortho), 2.02 (s br, 6H, CH₃ pyrrol), 2.10 (s, 6H, CH₃ Mes para), 6.08 (s, 2H, CH pyrrol), 6.68 (s, 2H, CH Mes), 6.71 (s, 2H, CH Mes), 6.84 (m, 6H, Cₘₑₜₐ—H P-Ph), 6.96 (m, 11H, Cₒᵣₜₕₒ—H P-Ph, Cₚₐᵣₐ—H P-Ph, Cₘₑₜₐ—H O—Ar), 7.05 (m, 1H, Cₚₐᵣₐ—H neophilydene Ph), 7.13 (m, 2H, Cₘₑₜₐ—H neophilydene Ph), 7.26 (m, 2H, Cₒᵣₜₕₒ—H neophilydene Ph), 7.60 (s br, 4H, Cₚₐᵣₐ—H B(Ar^{F})₄), 8.40 (s br, 8H, Cₒᵣₜₕₒ—H B(Ar^{F})₄), 11.02 (s, 1H, Mo═CH). ppm; ¹⁹F-NMR (282 MHz, C₆D₆): δ=−62.2 (B(Ar^{F})₄) ppm; ³¹P-NMR (121 MHz, C₆D₆): δ=22.8 (PPh₃) ppm; ¹³C-NMR (from HSQC and HMBC, 75 MHz, C₆D₆): δ=17.1 (CH₃ pyrrol), 19.5 (CH₃ Mes ortho), 20.2 (CH₃ Mes ortho), 20.6 (CH₃ Mes para), 30.3 (CH₃ ^{t}Bu), 29.5 (CH₃ neophylidene), 32.8 (CH₃ neophylidene), 52.3 (C neophylidene), 75.8 (C ^{t}Bu), 109.3 (CH pyrrol), 117.6 (d, ¹J_{CP}=100 Hz, Cᵢₚₛₒ P-Ph), 117.7 (Cₚₐᵣₐ—H B(Ar^{F})₄), 124.4 (q, ¹J_{CF}=265 Hz, CF₃), 126.2 (neophylidene Ph Cₚₐᵣₐ), 126.4 (neophylidene Ph Cₒᵣₜₕₒ), 127.8 (neophylidene Ph Cₘₑₜₐ), 128.6 (CH Mes), 129.4 (CH Mes), 129.6 (Cₘₑₜₐ P-Ph), 132.3 (Cᵢₚₛₒ Mes), 133.4 (NC pyrrol, Cₚₐᵣₐ P-Ph), 134.8 (Cₒᵣₜₕₒ—H B(Ar^{F})₄), 134.9 (Cₒᵣₜₕₒ P-Ph), 135.1 (Cₒᵣₜₕₒ Mes), 135.5 (Cₒᵣₜₕₒ Mes), 137.9 (Cₚₐᵣₐ Mes), 148.2 (neophylidene Ph Cᵢₚₛₒ), 162.2 (Cᵢₚₛₒ B(Ar^{F})₄), 164.5 (COMo), 291.2 pmp (¹J_{CH,SYN}=119.7 Hz, Mo═CH).

### Example 4: Synthesis of

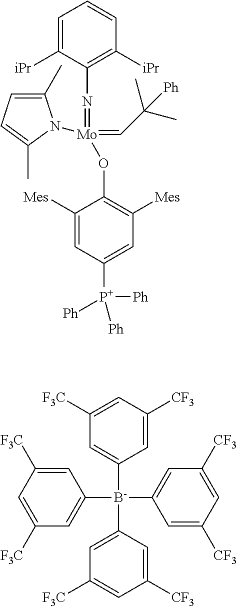

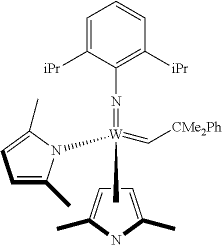

(10 mg, 0.017 mmol) was dissolved in benzene (1 mL) and

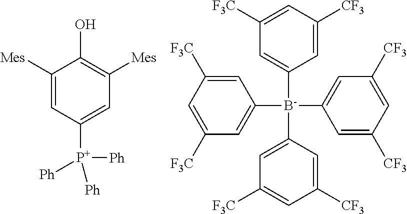

(25 mg, 0.017 mmol) was added as a solid. The suspension was stirred at room temperature for 12 h. The solvent was evaporated from the yellow solution and the resulting residue was taken in toluene (3 mL) which was subsequently evaporated. This operation was repeated 2 times to afford the target compound in 97% yield (32 mg) as a dark orange foam. ¹H-NMR (300 MHz, C₆D₆): δ=11.54 ppm (s, 1H, Mo═CH).

### Example 5: Synthesis of

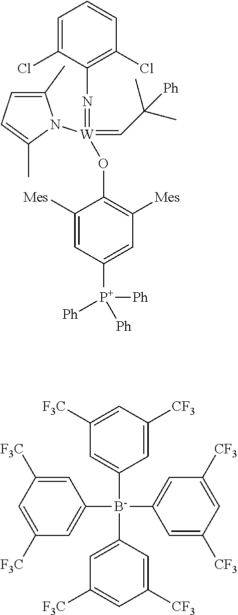

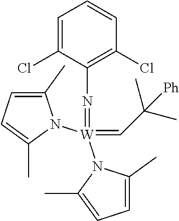

(10 mg, 0.015 mmol) was dissolved in benzene (1 mL) and

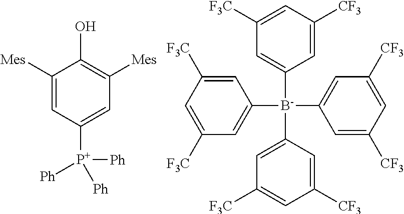

(22 mg, 0.015 mmol) was added as a solid. The suspension was stirred at room temperature for 12 h. The solvent was evaporated from the yellow solution and the resulting residue was taken in toluene (3 mL) which was subsequently evaporated. This operation was repeated 2 times to afford X823 in 98% yield (30 mg) as a dark orange foam. ¹H-NMR (300 MHz, C6D6): δ=8.49 ppm (s, 1H, W═CH).

### Example 6: Synthesis of

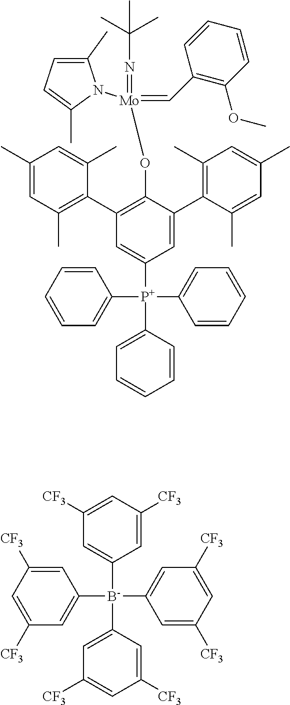

15 mg (8.12 μmol) of

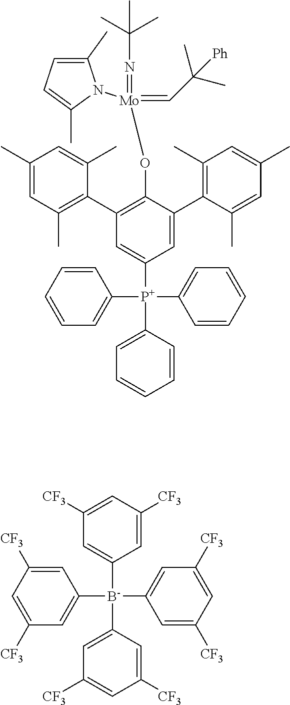

was dissolved in benzene (1 mL) and 1.3 mg 2-methoxy styrene

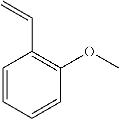

was added to the reaction mixture. It was stirred at room temperature for 16 h followed by evaporation. The precipitate was extracted with a mixture of n-pentane: diethyl ether (3:1 by volume) and the combined phases were concentrated to afford the title compound in 75% yield (11 mg) as a dark red foam. ¹H-NMR (300 MHz, C₆D₆): 5=12.43 ppm (s, 1H, Mo═CH).

### Example 7

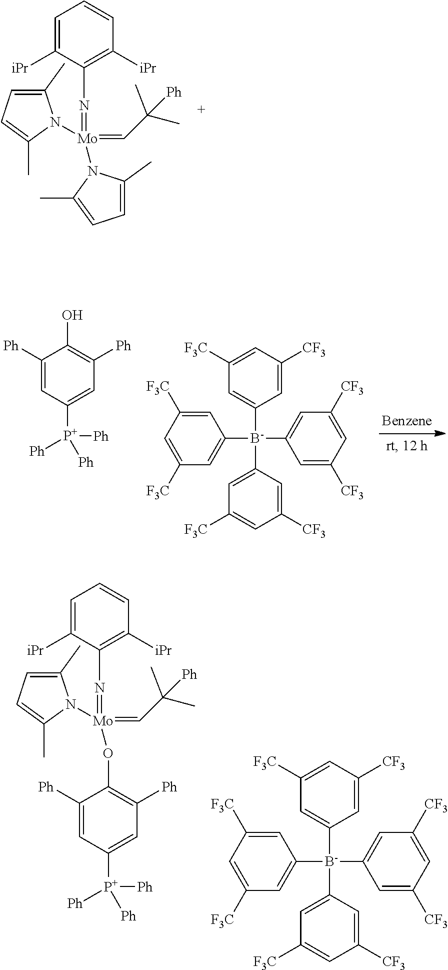

15 mg (0.025 mmol) of the bispyrrolide was dissolved in benzene (1 mL) and 35 mg (0.025 mmol) of phospohonium borate was added as a solid. The suspension was stirred at room temperature for 12 h. The solvent was evaporated from the yellow solution and the resulting residue was taken in toluene (3 mL) which was subsequently evaporated. This operation was repeated 2 times to afford the target compound in 95% yield (45 mg) as a dark orange foam. ¹H-NMR (300 MHz, C₆D₆): δ=11.36 ppm (s, 1H, Mo═CH).

### Example 8

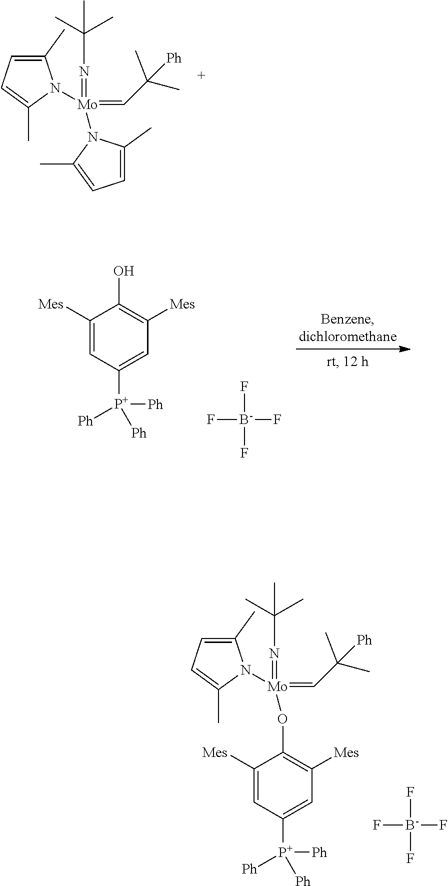

15 mg (0.031 mmol) of the bispyrrolide was dissolved in a mixture of benzene (0.6 mL) and dichloromethane (0.4 mL) and 21 mg (0.031 mmol) of the phosphonium borate was added as a solid. The suspension was stirred at room temperature for 12 h. The solvent was evaporated from the yellow solution and the resulting residue was taken in toluene (3 mL) which was subsequently evaporated. This operation was repeated 2 times to afford the target compound in 91% yield (30 mg) as a dark orange foam. ¹H-NMR (300 MHz, C₆D₆): δ=11.13 ppm (s, 1H, Mo═CH).

### Example 9

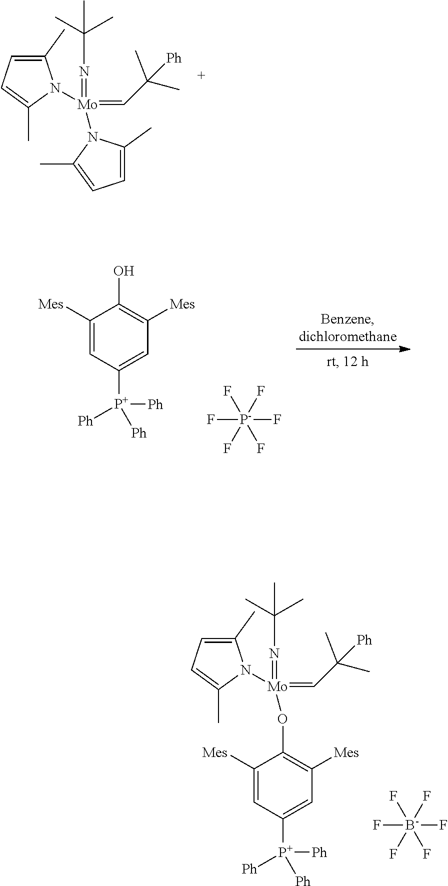

15 mg (0.031 mmol) of the bispyrrolide was dissolved in a mixture of benzene (0.6 mL) and dichloromethane (0.4 mL) and 23 mg (0.031 mmol) of the phosphonium phosphate was added as a solid. The suspension was stirred at room temperature for 12 h. The solvent was evaporated from the yellow solution and the resulting residue was taken in toluene (3 mL) which was subsequently evaporated. This operation was repeated 2 times to afford the target compound in 95% yield (33 mg) as a dark orange foam. ¹H-NMR (300 MHz, C₆D₆): δ=11.14 ppm (s, 1H, Mo═CH).

### Example 10

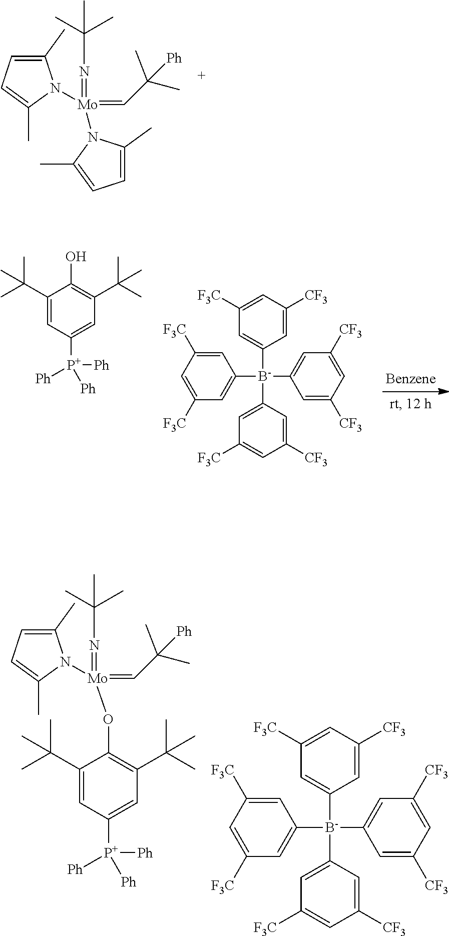

15 mg (0.031 mmol) of the bispyrrolide was dissolved in benzene (1 mL) and 41 mg (0.031 mmol) of the phosphonium borate was added as a solid. The suspension was stirred at room temperature for 12 h. The solvent was evaporated from the yellow solution and the resulting residue was taken in toluene (3 mL) which was subsequently evaporated. This operation was repeated 2 times to afford the target compound in 79% yield (42 mg) as a dark orange foam. ¹H-NMR (300 MHz, CD₂Cl₂): 8=12.85 ppm (s, 1H, Mo═CH).

Table 4 summarizes homo metathesis of 1-octene using the catalysts from Examples 2 to 10 under homogenous and heterogeneous conditions:

TABLE 4
HMC of 1-octene using various catalysts under
homogenous and heterogeneous conditions.

| | | | Loading | | Conversion | E/Z |
|---|---|---|---|---|---|---|
| Entry | Catalyst | Substrate | (ppm) | Media | (%) | ratio |
| 1 | Ex. 2 | 1-octene | 1000 | Homogeneous DCM | 26 | 6/94 |
| | | | | (dichloromethane) | | |
| 2 | Ex. 2 | 1-Octene | 1000 | Heterogeneous | 18 | 8/92 |
| | | | | Pyrrole/Heptane | | |
| 3 | Ex. 3 | 1-Octene | 1000 | Homogeneous DCM | 34 | 44/56 |
| 4 | Ex. 3 | 1-Octene | 1000 | Heterogeneous | 18 | 22/78 |
| | | | | Pyrrole/Heptane | | |
| 5 | Ex. 4 | 1-octene | 1000 | Homogeneous DCM | 33 | 14/86 |
| 6 | Ex. 4 | 1-Octene | 1000 | Heterogeneous | 48 | 34/66 |
| | | | | Pyrrole/Heptane | | |
| 7 | Ex. 5 | 1-Octene | 1000 | Homogeneous DCM | 39 | 7/93 |
| 8 | Ex. 5 | 1-Octene | 1000 | Heterogeneous | 25 | 11/89 |
| | | | | Pyrrole/Heptane | | |
| 9 | Ex. 7 | 1-Octene | 1000 | Homogeneous DCM | 90 | 83/17 |
| 10 | Ex. 7 | 1-Octene | 1000 | Heterogeneous | 78 | 61/39 |
| | | | | Pyrrole/Heptane | | |
| 11 | Ex. 6 | 1-Octene | 1000 | Homogeneous DCM | 71 | 50/50 |
| 12 | Ex. 6 | 1-Octene | 1000 | Heterogeneous | 75 | 18/82 |
| | | | | Pyrrole/Heptane | | |
| 13 | Ex. 8 | 1-Octene | 1000 | Homogeneous DCM | 73 | 26/74 |
| 14 | Ex. 8 | 1-Octene | 1000 | Heterogeneous | 63 | 13/87 |
| | | | | Pyrrole/Heptane | | |
| 15 | Ex. 9 | 1-Octene | 1000 | Homogeneous DCM | 79 | 60/40 |
| 16 | Ex. 9 | 1-Octene | 1000 | Heterogeneous | 77 | 19/81 |
| | | | | Pyrrole/Heptane | | |
| 17 | Ex. 10 | 1-Octene | 1000 | Homogeneous DCM | 37 | 31/69 |
| 18 | Ex. 10 | 1-Octene | 1000 | Heterogeneous | 2 | n/a |
| | | | | Pyrrole/Heptane | | |

### Example 11

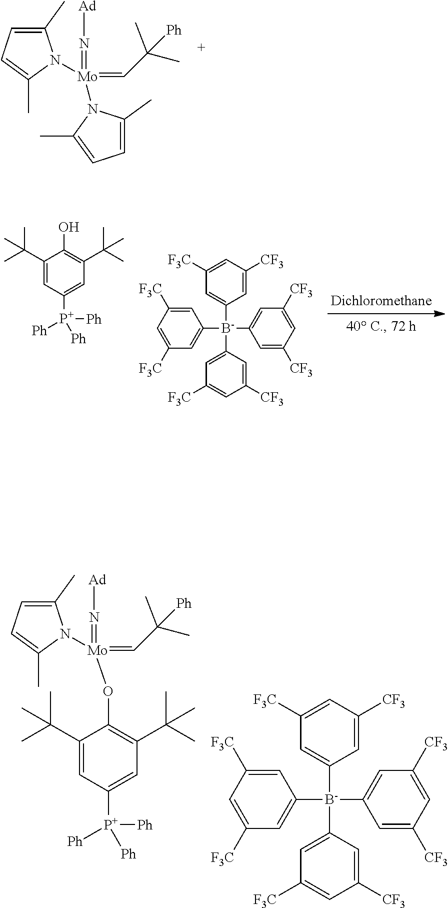

24 mg (0.042 mmol) of the bispyrrolide was dissolved in 1 mL dichloromethane and 56 mg (0.042 mmol) of the phosphonium borate was added as a solid. The suspension was stirred at 40° C. for 72 h. The solvent was evaporated from the yellow solution and the resulting residue was taken in toluene (3 mL) which was subsequently evaporated. This operation was repeated 2 times to afford the target compound in 98% yield (75 mg) as a dark brown foam. ¹H-NMR (300 MHz, CD₂Cl₂): δ=12.82 ppm (s, 1H, Mo═CH).

### Example 12

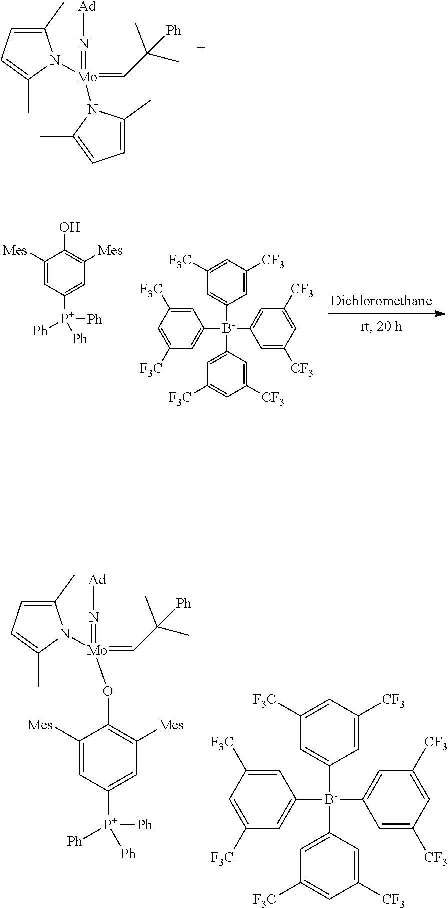

60 mg (0.106 mmol) of the bispyrrolide was dissolved in 5 mL dichloromethane and 154 mg (0.106 mmol) of the phosphonium borate was added as a solid. The suspension was stirred at room temperature for 20 h. The solvent was evaporated from the yellow solution and the resulting residue was taken in toluene (3 mL) which was subsequently evaporated. This operation was repeated 2 times to afford the target compound in 74% yield (150 mg) as a dark orange foam. ¹H-NMR (300 MHz, CD₂Cl₂): δ=10.95 ppm (s, 1H, Mo═CH).

Table 5 summarizes homo metathesis of various fatty acid methyl esters using the catalysts from Examples 11 and 12 under homogenous conditions:

TABLE 5
Homo metathesis of fatty acid methyl esters

| | | | Loading | | Conversion |
|---|---|---|---|---|---|
| Entry | Catalyst | Substrate | [ppm] | Media | [%] |
| 1 | Ex. 11 | Methyl cis-9- | 100 | Homogeneous | 45 |
| | | octadecenoate | | DCM | |
| 2 | Ex. 12 | Methyl 9- | 100 | Homogeneous | 31 |
| | | decenoate | | DCM | |
| 3 | Ex. 12 | Methyl cis-9- | 100 | Homogeneous | 50 |
| | | octadecenoate | | DCM | |

### Example 13

Synthesis of

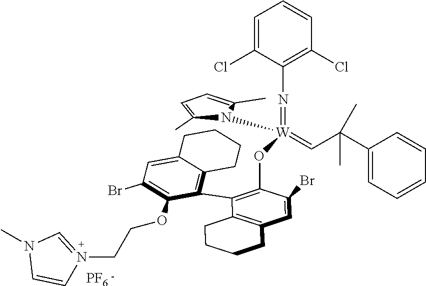

according to the following reaction scheme

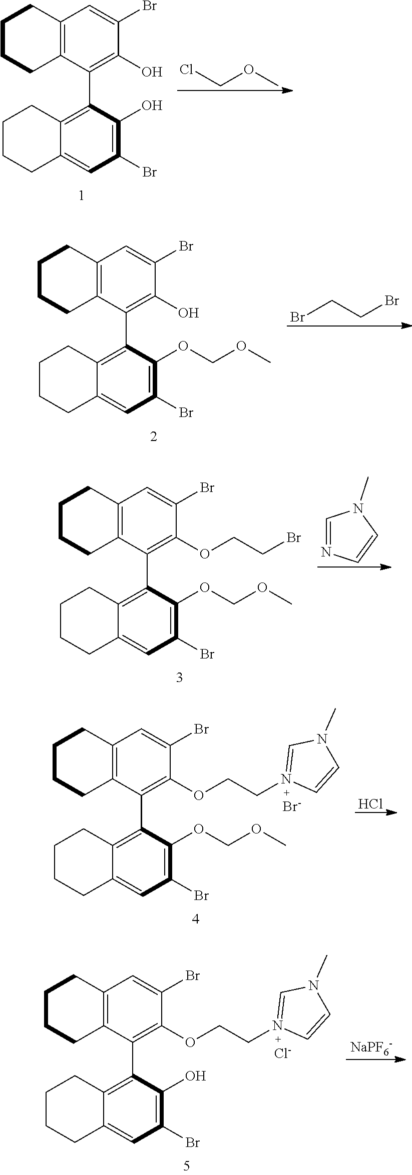

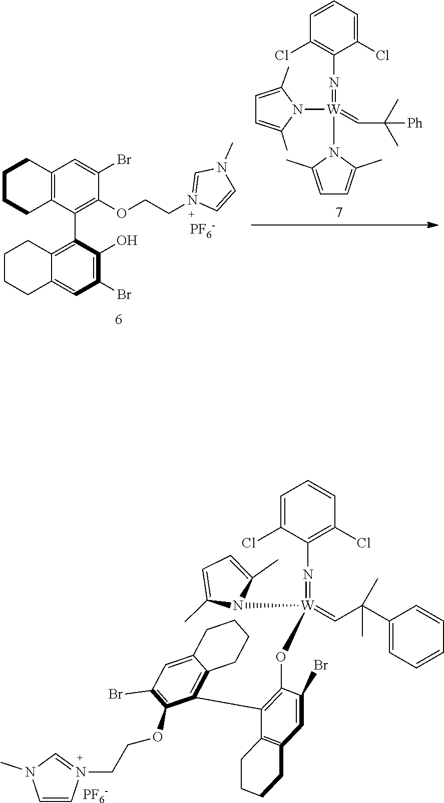

Synthesis of Compound 2

To the solution of starting material 1 (8.00 g, 17.7 mmol) in acetonitrile (70 mL) K₂CO₃ (7.34 g, 53.1 mmol, 3.0 equiv) was added, followed by chloromethyl methyl ether (1.42 g, 17.7 mmol, 1.0 equiv) at room temperature and the components were allowed to react for 16 h. Progress of the reaction was followed by TLC (heptane/EtOAc 5:1). The volatiles were removed in vacuo and the residue was partitioned in DCM/water mixture. The combined organics were dried over MgSO₄ and after evaporation of the solvent, the crude product was recrystallized from dichloromethane (5.95 g, 68%).

¹H NMR (300 MHz, CDCl₃): δ 7.37 (s, 1H); 7.23 (s, 1H); 4.88 (d, 1H), 4.76 (d, 1H); 2.94 (s, 3H); 2.79-2.69 (m, 4H); 2.40-2.27 (m, 2H); 2.08-1.92 (m, 2H); 1.76-1.55 (m, 8H).

Synthesis of Compound 3

To the solution of starting material 2 (2.00 g, 4.03 mmol) in acetonitrile (40 mL) K₂CO₃ (2.79 g, 20.2 mmol, 5.0 equiv) was added, followed by 1,2-dibromo ethane (2.27 g, 12.1 mmol, 3.0 equiv) and the resulting mixture was heated at 60° C. for 16 h. The volatiles were removed in vacuo and the resulting viscous mass was transferred to next step without further purification (1.99 g, 83%).

¹H NMR (300 MHz, CDCl₃): δ 7.34 (s, 1H); 7.29 (s, 1H); 4.90 (dd, 2H); 4.05-3.90 (m, 2H); 3.30-3.23 (m, 1H); 3.18-3.11 (m, 1H); 2.81 (s, 3H); 2.79-2.72 (m, 4H); 2.47-2.27 (m, 2H); 2.16-2.05 (m, 2H); 1.78-1.58 (m, 8H).

Synthesis of Compound 4

Mixture of compound 3 (500 mg, 0.83 mmol) and 1-methylimidazole (204 mg, 2.49 mmol, 3.0 equiv) were heated at 100° C. for 16 h. The volatiles were removed in vacuo and the residue was triturated in heptane and in EtOAc affording white crystals (450 mg, 79%).

¹H NMR (300 MHz, CDCl₃): δ 10.21 (s, 1H); 7.30 (s, 1H); 7.29 (s, 1H); 7.07-7.03 (m, 2H); 4.85-4.80 (m, 2H); 4.62-4.42 (m, 2H); 4.25-4.18 (m, 1H); 4.02-3.93 (m, 4H); 2.80 (s, 3H); 2.79-2.70 (m, 4H); 2.35-2.25 (m, 1H); 2.15-2.00 (m, 3H); 1.85-1.57 (m, 8H).

Synthesis of Compound 5

Compound 4 (2.00 g, 2.92 mmol) was added to mixture of DCM (30 mL) and 4M HCl in dioxane (6 mL) and resulting solution was agitated for 16 h. The volatiles were removed in vacuo and the residue was subjected to column chromatography (silica, DCM/MeOH 9:1) affording the title compound as white crystals (1.35 g, 77%).

¹H NMR (300 MHz, CDCl₃): δ 10.18 (s, 1H); 7.30 (s, 1H), 7.18 (s, 1H); 7.05-6.95 (m, 2H); 5.68 (br s, 1H); 4.55-4.35 (m, 2H); 4.20-3.90 (m, 5H); 2.80-2.65 (m, 4H); 2.25-2.05 (m, 2H); 2.05-1.85 (m, 2H); 1.85-1.50 (m, 8H).

Synthesis of Compound 6

Compound 5 (240 mg, 0.40 mmol) was added to mixture of DCM (3 mL) and NaPF₆ (67 mg, 0.4 mmol, 1.0 equiv) at room temperature and agitation was maintained for 16 h. The insolubles were removed by filtration and the filtrate was concentrated and transferred to the next step without further purification (259 mg, 92%).

¹H NMR (300 MHz, CDCl₃): δ 8.28 (s, 1H); 7.31 (s, 1H); 7.21 (s, 1H); 7.17 (s, 1H); 7.04 (s, 1H); 6.94 (s, 1H); 4.30-3.80 (m, 7H); 2.80-2.65 (m, 4H), 2.25-1.85 (m, 4H); 1.75-1.55 (m, 8H).

¹⁹F NMR (300 MHz, CDCl₃): δ−71.1 (s), −73.6 (s)

Synthesis of Target Compound

Compound 6 (230 mg, 0.33 mmol) was added to solution of compound 7 (216 mg, 0.33 mmol, 1.0 equiv) in DCM (10 mL) at room temperature and agitation was maintained for 16 h. The volatiles were removed in vacuo and the residue was triturated in pentane affording the title compound as a brown solid (198 mg, 47%).

¹H NMR (300 MHz, CDCl₃): δ 9.6 (s, 1H); 7.83 (s, 1H); 7.60 (d, 1H); 7.25-7.00 (m, 7H); 6.92-6.84 (m, 2H); 6.30-6.20 (m, 2H); 5.93 (s, 2H); 3.98-3.10 (m, 7H); 2.59-2.30 (m, 5H); 2.25-1.98 (m, 6H); 1.96-1.80 (m, 7H); 1.74-1.40 (m, 6H); 1.40-1.07 (m, 4H).

The target compound was tested in ethenolysis of methyl oleate resulting in methyl dec-9-enoate (9-DAME) and 1-decene. Table 6 shows results:

TABLE 6
Ethenolysis of methyl oleate

| Molar ratio | | | Yield | |
|---|---|---|---|---|
| Substrate/ | Conversion | Selectivity | 9-DAME | Turnover |
| catalyst | [%] | [%] | [%] | number |
| 667 | 82 | 0.7 | 62 | 411 |

9-DAME was subjected to homo metathesis using the target compound. Table 7 shows results. The reaction results in high Z selectivity.

TABLE 7
Homo metathesis of 9-DAME

| Loading catalyst | Conversion | |
|---|---|---|
| [ppm] | [%] | E:Z ratio |
| 250 | 76 | 10:90 |
| 100 | 44 | 5:95 |

1. A compound of formula I 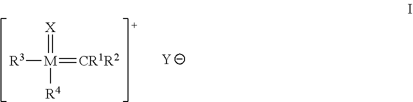
wherein:
M is selected from Mo or W;
X is selected from O or NR⁵;
R¹ and R² are independently selected from H, C₁₋₆ alkyl, and aryl; C₁₋₆ alkyl and aryl optionally being substituted with one or more of C₁₋₆ alkyl, C₁₋₆ alkoxy, and O—C₆H₅;
R³ is selected from a nitrogen-containing aromatic heterocycle being bound to M via said nitrogen; from halogen; and from triflate;
R⁴ is an aryl oxy group being bound to M via said oxygen of said aryl oxy group; wherein said aryl group Ar of said aryl oxy group is bound to a group Cat such to form a cationic ligand Cat⁺-Z—ArO—, wherein Z is either a covalent bond or a linker;
R⁵ is alkyl or aryl, optionally substituted; and
Y^{⊖} is a non-nucleophilic anion.

2. The compound of claim 1, wherein R¹ and R² are independently selected from H, C(CH₃)₃, C(CH₃)₂C₆H₅, and phenyl substituted in o-position with C₁₋₆ alkoxy.

3. The compound of claim 1, wherein R³ is selected from pyrrol-1-yl, pyrazol-1-yl, imidazol-1-yl, 1H-1,2,3-triazol-1-yl, 2H-1,2,3-triazol-2-yl, 1H-1,2,4-triazol-1-yl, 4H-1,2,4-triazo-4-yl, indol-1-yl, indazol-1-yl, and azaindol-1-yl, optionally substituted with one or more substituents selected independently from C₁₋₆ alkyl, C₁₋₆ alkoxy, phenyl, halogen, or cyano, preferably pyrrol-1-yl, 2,5-dimethylpyrrol-1-yl, and 2,5-diphenylpyrrol-1-yl or indol-1-yl or a substituted indol-1-yl.

4. The compound of claim 1, wherein R³ is selected from halogen.

5. The compound of claim 1, wherein said Ar in said Cat⁺-Z—ArO— is phenyl substituted in 2,6-position with phenyl, optionally substituted, or with isopropyl or t-butyl, respectively; or
said Ar in Cat⁺-Z—ArO— is phenyl substituted in 4-position with Cat⁺-Z—; or
said Ar in Cat⁺-Z—ArO— is phenyl substituted in 2,6 position with phenyl, optionally substituted, or with isopropyl or t-butyl, respectively; and
is substituted in 4-position with Cat⁺-Z—.

6. The compound of claim 1, wherein said group Cat forms together with Z—ArO— a group Cat⁺-Z—ArO— selected from an ammonium, pyridinium, phosphonium, phosphorinium, arsonium, sulfonium, and oxo sulfonium group, preferably
wherein said R⁴=Cat⁺-Z—ArO— is a pyridinium N-phenoxy group or a phosphonium P-phenoxy group.

7. The compound of claim 1, wherein said R⁴=Cat⁺-Z—ArO— is selected from the group consisting of 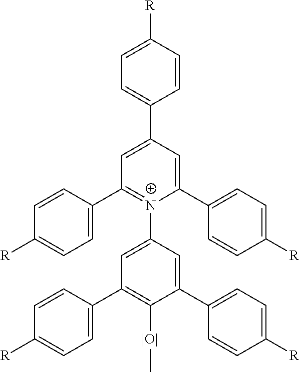
wherein R is H, C(CH₃)₃, CF₃, phenyl, or C₆F₁₃; 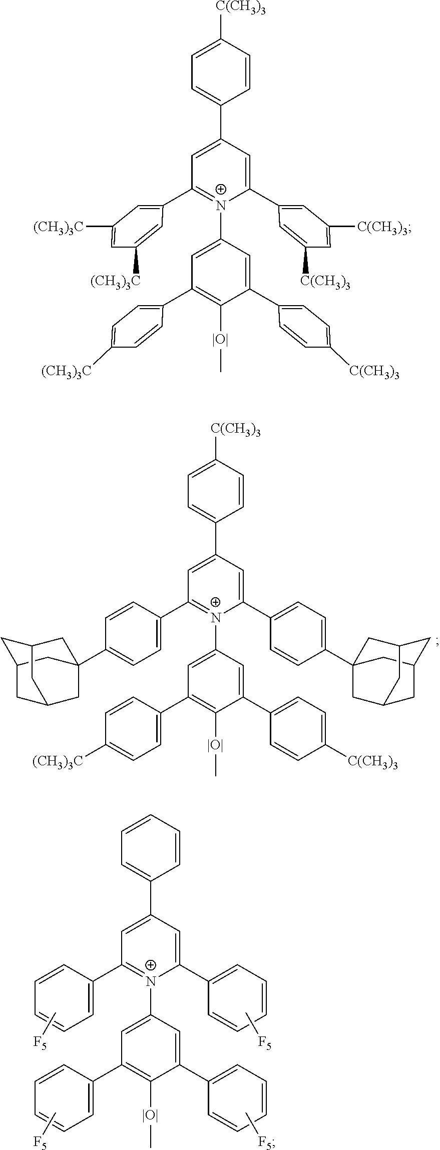 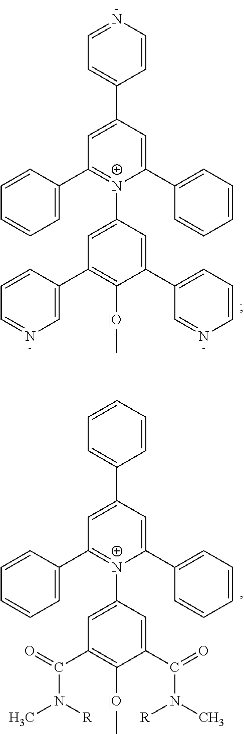
wherein R is H or CH₃; 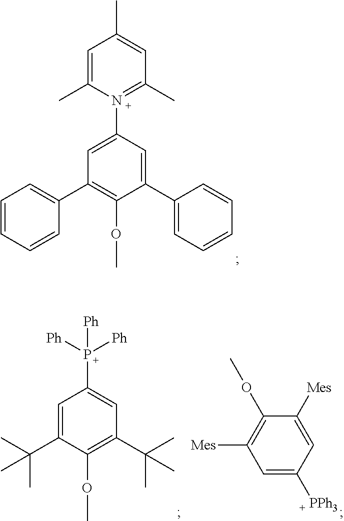
unsubstituted or substituted with C₁₋₁₀ alkyl, optionally substituted with halogen such as fluorine, C₁₋₁₀ alkoxy, nitro, cyano, phenyl, phenoxy, N(C₁₋₆ alkyl)₂, C(O)N(C₁₋₆ alkyl)₂, C(O)NH(C₁₋₆ alkyl), C(O)O—C₁₋₆ alkyl, halogen (F, Cl, Br, I) and two or more thereof 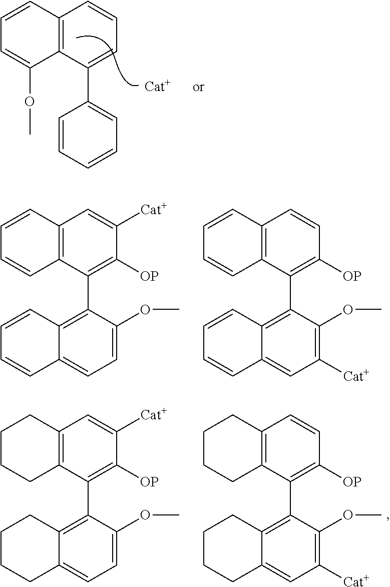
wherein P is a protecting group.

8. The compound of claim 1, wherein said non-nucleophilic anion Y^{⊖} is selected from ClO₄^{⊖}, AsF₆^{⊖}, SbF₆^{⊖}, PF₆^{⊖}, CH₃SO₃^{⊖}, CF₃SO₃^{⊖}, p-CH₃C₆H₄SO₃^{⊖}, BF₄^{⊖}, B[3,6-(CF₃)₂C₆H₃]₄^{⊖}, B[C₆F₅]₄^{Θ}, Al[O-t-C(CH₃)(CF₃)₂]^{⊖}, and Al[O-t-C(CF₃)₃]^{⊖}.

9. The compound of claim 1, wherein the compound of formula I is selected from the group consisting of:

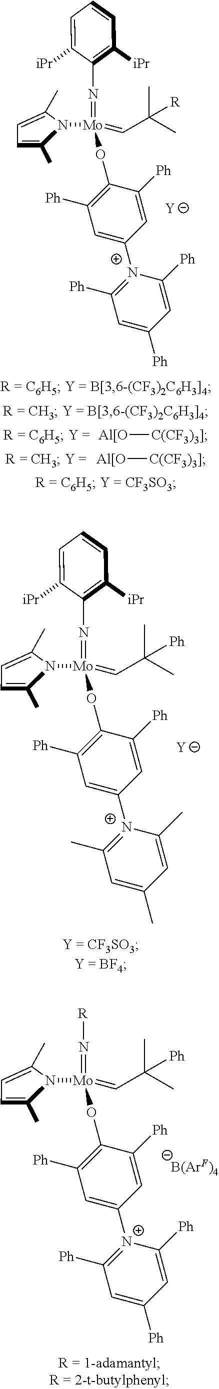

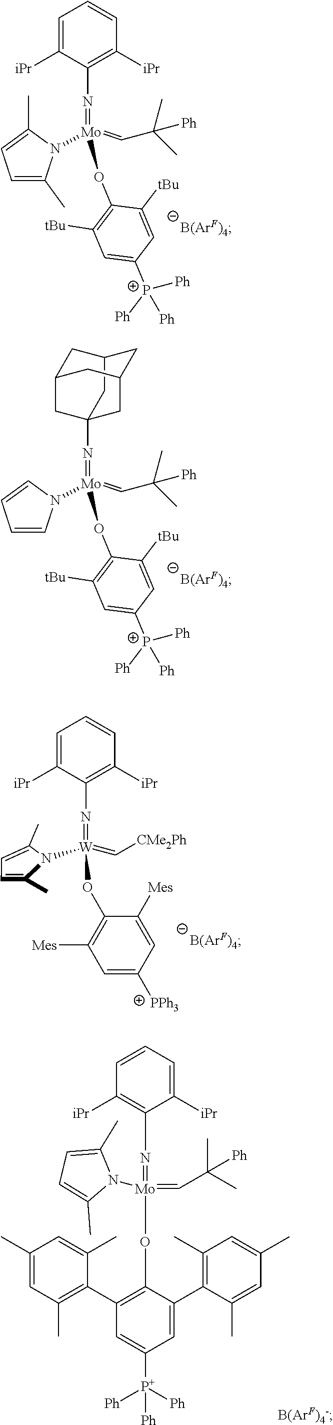

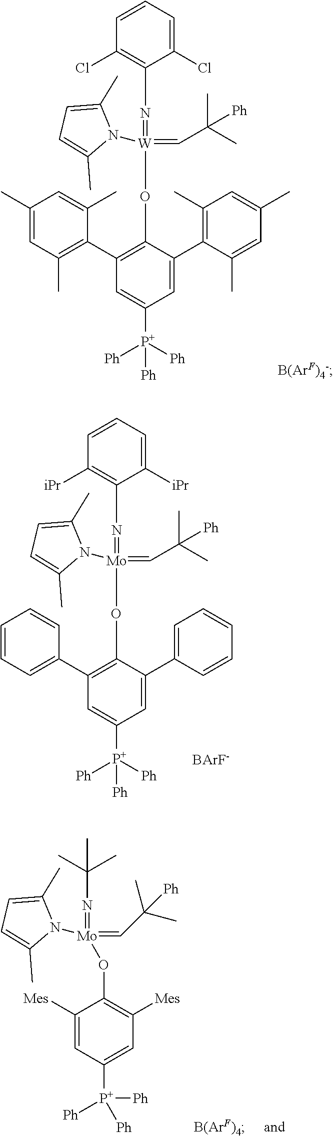

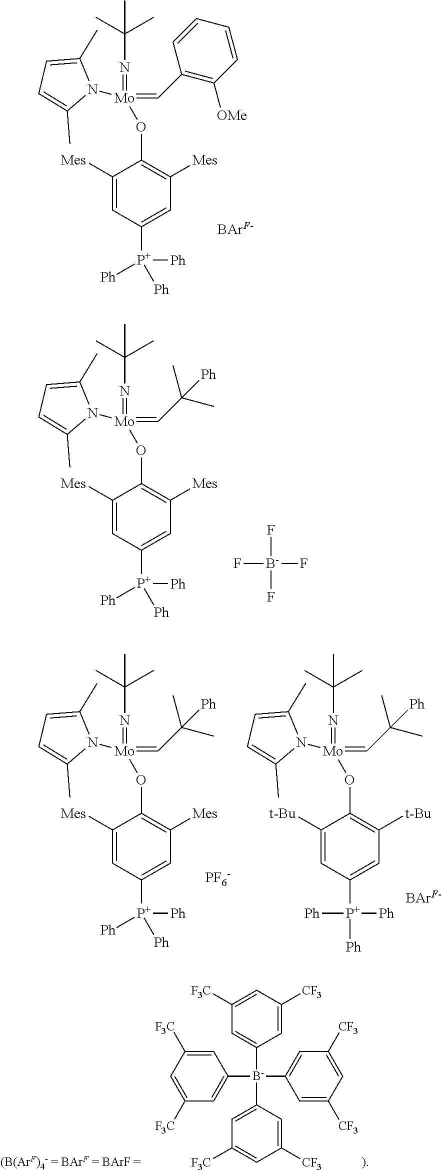

10. The compound of claim 1, wherein the compound of formula I does not contain a nitrogen-containing heterocyclic (NHC)-ligand.

11. A method of making a compound of formula I as defined in claim 1, the method comprising step (A):
(A) reacting a compound of formula II 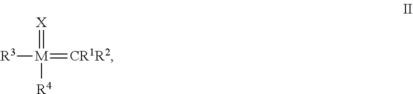
with a compound of formula III
[Cat⁺-Z—ArOH]⁺Y^{⊖} III,
wherein M, X, R¹, R², R³, Y^{⊖}, and Cat⁺-Z—ArO— have the meaning as defined in claim 1, and R⁴═R³,
to afford the compound of formula I.

12. A composition comprising a compound of claim 1, and a solvent; preferably wherein the solvent is selected from pyrrole, acetonitrile, dimethyl formamide, dimethyl sulfoxide, hexamethylphosphoramide, dimethylacetamide, and sulfolane, and an ionic liquid, or a mixture of two or more thereof, preferably wherein the ionic liquid is selected from

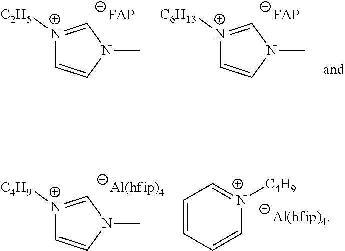

13. A method of performing a metathesis reaction, comprising step (B):
(B) reacting a first olefin with a second olefin, wherein the first olefin is identical to or different from the second olefin, in the presence of a compound as defined in claim 1.

14. The method of claim 13, wherein the metathesis reaction is performed in the presence of a composition comprising the compound and a further solvent, wherein the further solvent has a lower polarity than pyrrole, acetonitrile, dimethyl formamide, dimethyl sulfoxide, hexamethylphosphoramide, dimethylacetamide, and sulfolane or the ionic liquid such that said pyrrole, acetonitrile, dimethyl formamide, dimethyl sulfoxide, hexamethylphosphoramide, dimethylacetamide, and sulfolane or ionic liquid and the further solvent form two phases.

15. A method of performing a ring closing metathesis reaction
(a) comprising the use of a compound as defined in claim 1; or
(b) comprising the use of a composition comprising the compound of claim 1; or
(c) comprising the use of a composition comprising the compound of claim 1, and a further solvent, wherein the further solvent has a lower polarity than pyrrole, acetonitrile, dimethyl formamide, dimethyl sulfoxide, hexamethylphosphoramide, dimethylacetamide, and sulfolane or the ionic liquid such that said pyrrole, acetonitrile, dimethyl formamide, dimethyl sulfoxide, hexamethylphosphoramide, dimethylacetamide, and sulfolane or ionic liquid and the further solvent form two phases;
preferably wherein the ring closing metathesis reaction is a macrocyclisation.
